SEQUENCE LISTING (144 sequences)

SEQ ID NO: 1 - DNA, 2154 bp, synthetic construct
  source 1..2154: mol_type = other DNA; note = nucleotide sequence of synthesized PAL2
atggaccaaattgaggcaatgttgtgcggtggtggtgaaaaaacaaaggtcgcagtcacaactaaaacattagctgatccattgaactggggtttggctgctgaccaaatgaagggttctcatttagatgaagtcaaaaagatggtcgaggagtataggagaccagtcgtcaacttgggaggtgagacattgacaattggtcaagttgcagcaatttctactgtcggaggttcagttaaagtcgagttggctgagacatctagagctggagtcaaggcttcatcagactgggtcatggaatctatgaataaaggtacagattcatacggtgtcactactggtttcggtgctacttctcatagaagaacaaagaacggaacagctttgcaaacagaattgattagatttttgaatgctggtattttcggaaatacaaaagaaacttgtcatacattgcctcagtctgctacaagagctgctatgttggttcgtgtcaacacattgttgcaaggttattctggtataagatttgaaattttagaagctataacatctttattaaatcataatatttctccatctttgcctttgagaggtactattacagcatctggagatttagttccattatcatacattgctggtttgttgactggtaggcctaactcaaaggcaactggtcccgatggtgagtcattgactgctaaagaggctttcgaaaaagctggtatatctactggtttcttcgacttgcaaccaaaggagggtttggcattggtcaacggtactgctgttggttctggtatggcttcaatggttttatttgaagcaaatgttcaagctgttttggcagaggtcttgtctgcaattttcgctgaggttatgtctggtaagcccgaatttactgaccatttgactcatagattaaagcatcatcccggtcaaattgaagcagctgctattatggaacacattttggatggttcttcttacatgaaattggctcagaaggttcatgagatggacccattgcagaagccaaagcaagataggtacgctttgagaacttcaccacagtggttgggtccacagattgaagttataagacaagctactaaatctatagaaagagaaattaattcagttaacgataatccattgattgatgtttcaagaaataaggctattcatggtggtaattttcaaggtactccaattggtgtttctatggataatactagattggctattgcagcaattggtaagttaatgtttgcacaattctcagaattagttaacgatttttataataacggtttgccatctaatttaactgcttcttctaatccttctttggattacggatttaagggtgctgaaatagctatggcttcatattgttctgaattgcagtacttagctaatcccgttacttctcacgttcagtcagctgaacagcacaaccaagatgttaactctttgggtttgatatcatctagaaaaacatcagaagctgttgatattttgaaattgatgtctactactttcttggtcggtatttgtcaagctgtcgacttgaggcatttggaggaaaatttgagacagacagttaaaaacactgtctcacaagttgctaagaaagtcttgacaactggtattaatggtgaattacatccatctaggttttgtgaaaaggacttgttaaaggttgttgacagagaacaagttttcacatacgtcgacgacccatgctcagctacttacccattgatgcaaagattaagacaagttatagttgatcacgctttatctaatggtgagacagagaagaacgcagttacatctatttttcaaaaaattggtgctttcgaagaagaattgaaagcagttttgccaaaggaagttgaggctgctagggctgcttacggaaatggtacagctccaattcctaatagaattaaagaatgtcgttcttacccattatacagatttgtcagagaagaattaggtacaaagttattgactggtgaaaaggtcgtctcacccggtgaggagtttgacaaagtttttactgctatgtgtgagggtaaattaattgaccctttgatggactgcttgaaagagtggaacggtgctcctattccaatttgttaa

SEQ ID NO: 2 - DNA, 1518 bp, synthetic construct
  source 1..1518: mol_type = other DNA; note = nucleotide sequence of C4H
atggacttgttgttgttggaaaagtcattaattgctgtttttgttgctgttattttggctactgttatttctaaattaagaggtaaaaagttgaaattgccacccggtccaattcctattccaatttttggtaattggttgcaagtcggtgacgatttgaatcaccgtaatttggttgattacgctaaaaagtttggtgacttatttttattgagaatgggtcaaagaaatttagttgttgtttcttctccagatttgactaaggaagttttattgacacaaggtgttgaattcggttcaaggactagaaatgttgttttcgatatttttactggtaaaggtcaagacatggtttttacagtttatggtgaacactggagaaaaatgaggagaattatgactgttccatttttcacaaataaggtcgtccagcagaatcgtgaaggatgggagtttgaagctgcatctgtcgtcgaagacgtcaagaagaacccagattctgctactaaaggtattgttttgaggaagagattgcaattgatgatgtatactaatatgtttaggataatgtttgatagaagattcgaatctgaagatgatcctttgttcttgagattgaaggctttgaatggtgaaagatctagattagcacaatcttttgaatacaattacggtgacttcataccaattttgagaccatttttaagaggttacttgaagatttgccaagatgttaaggatagaagaattgctttatttaaaaagtacttcgttgatgaaagaaaacaaattgcttcttctaagccaactggttctgaaggtttaaaatgtgctattgaccatattttggaagcagagcaaaagggtgaaattaatgaagataatgttttgtacatagttgaaaatataaacgttgctgctattgagactacattgtggtctattgaatggggtattgctgaattagtcaaccaccccgaaattcaatctaagttgggtaacgaattggacactgtcttgggtcccggtgttcaagtcacagagccagacttgcataaattaccttatttgcaagcagtcgtcaaagaaactttaagattgaggatggctattccattgttggttcctcatatgaatttacatgatgctaagttggctggttacgacatacccgctgagtctaaaattttggttaatgcatggtggttggctaacaacccaaactcttggaagaagccagaggaatttcgtccagagaggttcttcgaagaggagtctcacgttgaggctaacggtaacgactttcgttacgtcccattcggtgttggtaggagatcatgtcccggtattatattagctttgccaattttgggtattactataggtagaatggtccaaaactttgagttgttgccaccacccggtcagtctaaggtcgacacttctgagaagggtggtcaattttcattacatattttgaaccattctattattgttatgaagccaagaaattgttaa

SEQ ID NO: 3 - DNA, 1686 bp, synthetic construct
  source 1..1686: mol_type = other DNA; note = nucleotide sequence of 4CL2
atgattactgcagcattgcacgagccacagattcacaagccaactgacacttctgtcgtctctgatgatgtcttgcctcactctccacctactccaaggattttcagatcaaaattgcccgatattgatattcctaatcacttaccattgcatacatactgtttcgaaaaattgtcttcagtctcagataagccatgtttgattgttggttcaactggtaaatcttacacttacggtgaaactcacttgatttgtcgtagggtcgcttctggtttgtacaagttgggaataagaaaaggtgatgttattatgattttgttacaaaattcagcagaattcgtcttctcatttatgggtgcttctatgattggtgctgtttctactacagctaacccattttatacatctcaagaattatataagcagttgaaatcttctggtgctaagttgataattactcactctcagtacgttgataaattgaagaatttaggtgaaaatttgacattgattactactgatgaaccaacacccgaaaattgcttgccattctctactttgattactgacgatgagactaacccattccaagaaactgtcgacattggtggtgacgacgcagctgcattaccattttcttctggtactactggtttaccaaaaggtgttgtcttgactcataagtctttaattacatctgtcgctcagcaagttgatggtgataacccaaacttatatttgaaatcaaatgatgttatattatgtgttttgccattatttcatatttactctttgaattcagttttgttaaattcattgagatctggtgctactgttttgttaatgcataagtttgaaataggtgctttgttggatttgattcaaagacatagagtcactattgcagctttggttccaccattggtcattgctttggctaaaaaccctacagttaattcttatgatttgtcatctgtcagattcgtcttgtctggtgctgcaccattgggaaaggagttgcaagattctttgaggaggaggttgccacaagctattttgggtcaaggttacggtatgacagaagctggtccagtcttgtctatgtctttgggattcgctaaagaacctattccaactaagtctggttcttgcggtacagttgtcagaaacgctgagttgaaggttgttcatttggaaacaaggttgtctttgggttataaccagcccggtgaaatatgtattagaggacaacaaattatgaaggaatatttaaacgatccagaagctacttctgctactattgacgaggagggttggttgcatactggtgacattggttacgtcgacgaggatgatgaaatattcattgttgatagattaaaagaagttattaaatttaagggttttcaagttccaccagctgagttggagtctttgttaattaatcatcactctattgctgatgctgcagtcgttccacagaacgatgaagttgctggtgaagtccccgttgctttcgtcgttaggtctaatggtaacgatattacagaggaagatgttaaagaatacgtcgctaaacaagttgttttttacaaaagattgcataaagttttcttcgtcgcatcaattcctaaatctccatctggtaaaattttgagaaaggatttaaaagctaagttatgttaa

SEQ ID NO: 4 - DNA, 1308 bp, synthetic construct
  source 1..1308: mol_type = other DNA; note = nucleotide sequence of HCT
atgaaaattgaagttaaagaatctactatggtcaagccagcagctgagacaccacagcaaagattgtggaactctaatgttgatttggttgtcccaaattttcatacaccatctgtttatttttacagacctactggttctccaaacttttttgatggaaaagttttaaaggaagcattgtctaaagctttggtccctttctacccaatggctggtaggttgtgcagagacgaggatggtagaattgagattgattgtaagggtcaaggtgttttgttcgttgaggcagagtcagatggtgttgtcgacgacttcggtgactttgctccaacattggagttgaggcagttgattcccgctgtcgactactctcaaggtattcaatcatacgctttattggttttgcaaattactcactttaagtgcggtggtgtctctttgggtgttggtatgcagcatcatgcagctgatggtgcttctggtttgcactttattaatacatggtctgatatggctagaggtttggatttaactattccaccttttattgatagaacattgttaagagctagagacccaccacagccacaattccctcacgtcgagtatcagccaccacctactttgaaggtcactccagagaatacacctatttctgaagctgttccagaaacatctgtctctatatttaaattgactagagatcaaattaacacattgaaagcaaaatctaaagaagatggtaacacagttaattactcttcttatgaaatgttggctggacacgtctggagatctacatgcatggctaggggtttggctcacgaccaagaaactaaattgtatattgctacagacggtaggtctagattgaggccatctttgccacccggttactttggtaacgttatttttactactactcctattgctgttgctggtgatattcaatctaaaccaatttggtacgctgcttctaagttgcatgatgcattggctagaatggataatgactatttgagatctgcattggattacttggagttgcaaccagacttgaaggctttagttcgtggtgctcacactttcaagtgcccaaacttgggtattacttcttggtctagattacctattcatgatgctgatttcggttggggtaggccaattttcatgggtcccggtggtatagcttacgaaggtttatctttcattttaccatctccaactaatgatggttctcaatctgttgctatttctttacaagctgaacatatgaaattgtttgagaaatttttgtacgatttttaa

SEQ ID NO: 5 - DNA, 720 bp, synthetic construct
  source 1..720: mol_type = other DNA; note = nucleotide sequence of CCoAoMT1
atggctactaacggtagacaccaagaagttggtcacaagtctttgttgcagtctgatgcattgtatcaatatattttggaaacatctgtctacccaagagagccagagcctatgaaggagttgagagaaattacagctaaacatccatggaatttgatgactacttctgctgatgaaggtcaatttttgtctatgttgattaagttgattaacgctaaaaatactatggaaattggtgtttttactggttactcattgttggctacagctatggcattgccagatgatggtaaaatattagctatggatattaatagagaaaattatgaaattggtttgccagttattgaaaaagctggtttggctcataaaattgagtttaaagaaggtccagctttgccagtcttggaccagatgatagaagatggtaaatatcacggttcttatgattttattttcgttgatgctgacaaagataattatttaaactatcacaaaagattaattgatttggttaaaattggtggtttgattggttatgataacactttgtggaatggttctgtcgttgctccaccagatgcaccattgagaaagtacgttagatattatagagattttgttttagaattaaataaagcattggctgctgattctagaattgaaatttgccagttgccagtcggtgacggtataactttgtgcagaagaatatcttaa

SEQ ID NO: 6 - DNA, 834 bp, synthetic construct
  source 1..834: mol_type = other DNA; note = nucleotide sequence of FerB2
atgtctgatgaattgacatttgaaactgttaaagttactttggacgacggtattgcttgggttgctttgaacagaccagagaagaggaacgctatgtctccaactgtcaacagagagatgtgggtcgccttggacgagttggagattagagacgacgtcggtgtcgtcgttttgactggtgaaggtgaggctttctctgctggtatggacttgaaggaatactttcgtgagactgagggtgaatctgctattgtccaagctaggtatcgtagagctgctactgactggatgtggagaagattgatgcattacccaaagccaactattgctatggtcaacggttggtgcttcggtggtgctttcactccattggtcgcttgtgacttggctattgctgcagacgaagctactttcggtttgtctgaaattaattggggtattattccagctggtaacgttactagagctgtcgctcaagttatgaatcatagagacgctttgcactatattatgactggtgatcctttcgacggtaaggaagctgctaagatgggtttggttaatagatcagttccattagcagaattaagaggtgaagttgaagttttggctagaaaattgttatctaaaaatccaactattttgagacatgctaaatctgcatttaaatatgtcgaaagattggactgggacacttctgaagctatgttgggtgctttggctgcaacagctgcttctcaagatactgagaggggtagaaggaagggtatgacacaatttttggatgaaaagtcttttaaacccggtttgggtggttatagaagagaagattaa

SEQ ID NO: 7 - DNA, 1380 bp, synthetic construct
  source 1..1380: mol_type = other DNA; note = nucleotide sequence of pAMT
atggcaaatattacaaacgaatttatgggtcatgatatgttagcaccattcacagctggttggcagtcagacatggaaccattggttatagaaaagtctgaaggttcttacgtctatgatattaacggtaaaaagtacttggatacattgtctggtttgtggtgtgctactttgggtggttctgagacaagattggtcgaagctgctaataaacaattaaacactttaccattttaccactctttttggaacagaacaacaaaaccttctttggatttagctaaagagttattgaatatgtttactgctaataaaatggctaaggtcttttttacaaactctggttctgaagctaatgatacacaagtcaagatggtttggtattataataatgctttgggtagacctcagaagaagaaaattattgctagagcaaaagcttatcacggttcaacatacatttctgctggtttatctggtttgcctccaatgcaccagaagttcgacttgccaccaccattcgtcttgcatacagaatgcccacactactgggcataccatttgcccggtgaaacagaagaagaattctctactagattggcaaataatttggaatctttaattttgaaagagggtccagaaactgtcgctgcattcattgctgaaccagtcttgggtgcagctggtgtcattttaccaccagctacttattttgataaggttcaagcaattttgagaaagcatgatattttgtttattgctgatgaggttgtttgcggtttcggtagattgggaactatgtttggttctgataagtataacattaagccagacttggtctcagtcggaaaggcattgtcttctggttatatgccaattgctgcagttttagtttcacaaaaaatttcttctgtcattttatctgaatctaataaaataggtgctttttgtcatggtttcacatactctggtcatccagttgcttgcgctgttgcattggaggctttgaaaatttacaaagaaaggaatattacagaagttgtcaataaaatttcacagaaattccaagaaggtttgaaagcttttgctgattcaccaattattggtgaaataagaggtactggtttggctttgtctactgagttcgttgataataaatcaccaaatgatcctttcccatatgaatgggctgtcggtacttacttcggtgctcagtgcgctaagtacggtatgttggtctcatctactggtgaccacgtcaacatggctccaccatttatattgtctttggaggaattggatgagttaattagaatatacggtaaggctttaaaagatacagaaaaaagagttgaagaattgaagtcacaaaaacaataa

SEQ ID NO: 8 - DNA, 804 bp, synthetic construct
  source 1..804: mol_type = other DNA; note = nucleotide sequences of TDH3p
atactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaagatg

SEQ ID NO: 9 - DNA, 622 bp, synthetic construct
  source 1..622: mol_type = other DNA; note = nucleotide sequences of ADH1p
acatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacagaat

SEQ ID NO: 10 - DNA, 750 bp, synthetic construct
  source 1..750: mol_type = other DNA; note = nucleotide sequences of PGK1p
acgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 11 - DNA, 562 bp, synthetic construct
  source 1..562: mol_type = other DNA; note = nucleotide sequences of TEF2p
tatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacactagtacc

SEQ ID NO: 12 - DNA, 405 bp, synthetic construct
  source 1..405: mol_type = other DNA; note = nucleotide sequences of TPI1t
acctgattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactcataagtgtaccgaagactgcattgaaagtttatgttttttcactggaggcgtcattttcgcgttgagaagatgttcttatccaaatttcaactgttatatag

SEQ ID NO: 13 - DNA, 405 bp, synthetic construct
  source 1..405: mol_type = other DNA; note = nucleotide sequences of PGI1t
atcgaacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaagatgaatcagtgcgcgaaggacataactcatgaagcctccagtatacc

SEQ ID NO: 14 - DNA, 158 bp, synthetic construct
  source 1..158: mol_type = other DNA; note = nucleotide sequences of ADH1t
agttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccga

SEQ ID NO: 15 - DNA, 248 bp, synthetic construct
  source 1..248: mol_type = other DNA; note = nucleotide sequences of CYC1t
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 16 - DNA, 520 bp, synthetic construct
  source 1..520: mol_type = other DNA; note = nucleotide sequences of ENO2p
acgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 17 - DNA, 290 bp, synthetic construct
  source 1..290: mol_type = other DNA; note = nucleotide sequences of PYK1p
aggagacgttactttgtttatatatattagtatgtacaatcgcaaagaaatggagtgatgacatgttgtagtatttagtatgaggttactgtgtgggaggttttaccatgatttttggcgagaacacgccatgaaatgtctttgtacgaaactcattacccgcattaatattttttttctttttaaagctcagttgaccctttctcattcccttcttaaaacaactgtgtgatccttgagaaaagataaattacatacacaacataaacccaactacgatcgcaaattat

SEQ ID NO: 18 - DNA, 630 bp, synthetic construct
  source 1..630: mol_type = other DNA; note = nucleotide sequences of FBA1p
ataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 19 - DNA, 469 bp, synthetic construct
  source 1..469: mol_type = other DNA; note = nucleotide sequences of HOG1t
tttaatgtccctaaccactcattcttacttctttttgatgtttcttttttttatggtactcataaaagtatttacgtatatagttgtatagaggaaacaaaaaaaaaaagataaaactcaattacaaagtaaagtggacgtatttcgatcatgatttttttctgttttaaccgcattggattttcttgtaaaactggaagaaaaaggaaactaaaaagtcaagaaagacctttttaagactccaagaaccgtcacttatggcgtattgtttgtttatcagcacttctatcttcgataaaggtttgtctgtcttatattgtttacatttcaagtctaattctgtgcttttaccgaagaggaattttcataaatacggagaaaatataaaaaaaaagtaaatacagaaaatagaacagttgaagcagaaaaagagaacttgctaaatagctgtctcacccagacaagcgta

SEQ ID NO: 20 - DNA, 438 bp, synthetic construct
  source 1..438: mol_type = other DNA; note = ucleotide sequences of LRP1t
aggtcgacgtatactggtacatgagtgaaaaaatgattaaaggtgatgtgagagcacaaatgcaaaaatgcttatagggtttatatacttttataataacaataagaactcgttgaagtaactaatatgtgtatatgctattcttgtagttggcagtgaacacttttgtgtgaattgacgttatataagactgtagaaatatctaagaaaagagagttttcataaaacccttcaagcttaactaccaaaaagcattcatacttagaagttgaaatgcacgatgatgccaaattgatgaaaaaagctaaaaatgcatttcagaagggaagaaaataataagcaacataacagagggaataggtgcgcttagtcttgttgctgtaaaattaaccaattattcttgtattgttctaacgatccgaccatttcatcctgaat

SEQ ID NO: 21 - DNA, 458 bp, synthetic construct
  source 1..458: mol_type = other DNA; note = nucleotide sequences of ATP15t
tttaacgcttcctgggaactgcagctctttttttactcgctgatatacattttaaatattctagcaactgtgtatgaaaacttacgtacttttatacgggaaactaataatgactacaatgatattgaatactggccgcttcgaagagtggtataaagtttgtatcattgcattaaaagaaaaagaaatatatgtcccatcatcgccaatcgcaatgttgaatggtcgtttaccacttttgcggctgggcatatgcagaaacatgctgtcccgtccccgactggctaaactgccatctataaggtttcggtctttggtcaccccttcttcatcgcagctcattcctctcagtcggttgtgtttaaggtcacctgcagttggaaaatcactaattttacaaagttttagatgtaattcatccaaaacagttccagaaacttctctgccttcagcctctc

SEQ ID NO: 22 - DNA, 1161 bp, synthetic construct
  source 1..1161: mol_type = other DNA; note = nucleotide sequence of coding gene pheA
atgacatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccaccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcaaatcagcgacaaaacttcacccgatttgtggtgttggcgcgtaaagccattaacgtgtctgatcaggttccggcgaaaaccacgttgttaatggcgaccgggcaacaagccggtgcgctggttgaagcgttgctggtactgcgcaaccacaatctgattatgacccgtctggaatcacgcccgattcacggtaatccatgggaagagatgttctatctggatattcaggccaatcttgaatcagcggaaatgcaaaaagcattgaaagagttaggggaaatcacccgttcaatgaaggtattgggctgttacccaagtgagaacgtagtgcctgttgatccaacctga

SEQ ID NO: 23 - DNA, 1542 bp, synthetic construct
  source 1..1542: mol_type = other DNA; note = nucleotide sequence of transaminase coding gene Aro9
atgactgctggttctgccccccctgttgattacacttccttaaagaagaacttccaaccgtttctctccagaagagtagaaaatagatctctgaaaagcttttgggatgcttctgatatctcagatgacgtcattgagctagctggtggaatgccaaacgagagattttttcctatcgaatctatggatttgaaaatatcaaaagttccttttaatgataacccaaaatggcataattcgtttaccacggcgcatttggacttgggatcccccagtgagctacccattgcacgttctttccaatatgcagaaaccaagggtttaccccctctcttacattttgttaaagattttgtgtccagaattaatcgcccagccttttccgatgagacggagtctaactgggatgtcatcctttctggcgggtccaacgattcaatgtttaaggtttttgaaacaatttgcgacgaatcgaccactgtgatgattgaagagtttactttcaccccggctatgtccaatgtggaggctacaggagcaaaagtcatccccatcaagatgaacctgaccttcgacagagagtcccagggtattgatgtcgaatatctaacgcagttgctcgataattggtcaactggaccatacaaagacttaaacaagccaagggtcctatataccattgcaacgggccaaaatcctaccgggatgtctgtcccccagtggaaaagagagaaaatttaccagttggcccaaagacacgatttcctcattgttgaagatgatccctacggttatctgtactttccttcctataatccgcaagagccattagaaaacccttaccattctagcgacctgactactgaacggtatttgaatgattttttaatgaaatcattcttgactttggatacagatgcccgtgtcatccgtttggagactttttctaaaatttttgctcctggattaaggttatccttcatcgttgctaataaattccttttgcaaaaaatcttggatttggccgacattactacaagggcccccagtggtacctcacaagctattgtttattctacaataaaggcaatggctgagtccaacttatcgtcctctctttctatgaaagaagcaatgtttgagggttggataagatggataatgcagattgcttctaaatacaatcataggaaaaatcttactttgaaagccttatacgaaacagaatcttaccaagctggtcagtttaccgttatggaaccctccgcgggtatgttcatcattattaaaatcaattgggggaatttcgatagacctgacgatttgccgcaacagatggatattttagataagttcttgctgaagaatggtgttaaagtagtgcttggttataaaatggctgtttgcccaaattattcaaagcagaattcagattttctaagactcaccatcgcctatgcaagggatgatgatcagttgattgaagcttccaaaagaatcggtagtggcataaaagaattttttgacaactataaaagttga

SEQ ID NO: 24 - DNA, 290 bp, synthetic construct; identical to SEQ ID NO: 17
  source 1..290: mol_type = other DNA; note = nucleotide sequence of PFY1p promoter

SEQ ID NO: 25 - DNA, 669 bp, synthetic construct
  source 1..669: mol_type = other DNA; note = nucleotide sequence of ACT1p promoter
acaagcgcgcctctaccttgcagacccatataatataataactaaataagtaaataagacacacgcgagaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaaca

SEQ ID NO: 26 - DNA, 408 bp, synthetic construct
  source 1..408: mol_type = other DNA; note = nucleotide sequence of PRC1t terminator
taaagcgtgtatgtgtaggcataccgtttttattatcagctacgatcgaaatatatacgtttttatctatgttacgttatatattgtagtcttaaccatttgtaagttttgcttcttttcttttattcatttcataacatcatactgttttacgtaataccatccttaactttttccaccgaaggggaggaagaactaaattctagaaaaatgaaaccattaaaaaaaaagaaaacaatagagctgcttctacaattttgcacatcaaaaaagacctccaactaccgagtttgtaagtacaatgtctcaacccactcccatcataactacaaaatcagctgctaagccaaaaccaaaaatttttaatttattccgcgtttgcttcatttcattattgctgatcgctgc

SEQ ID NO: 27 - DNA, 416 bp, synthetic construct
  source 1..416: mol_type = other DNA; note = nucleotide sequence of BAN4t terminator
agccagtttattcttgccatccgtgtacgctaggagaggattattaaaataagtgatatatacatatatatatatatatatatatatataatacactaattattttatgtgatgttgatcacgcgaaacggtaaacggctctgttcgcgctttctttgtttacattttagtgaagtattgtcaagataatatccatatggttgaactttttatattggctagttaaatactctatttattgcaccaaaaaatcatcttagtggactttttggagcaaaaaaaaccagcaggtagttatataagatgactacacaactaaggtatgaaaataacgatgacgatgaaagagtagaatataatctctttaccaatagatccaccatgatggcaaattttgaagaatggatcaaaatggc

SEQ ID NO: 28 - DNA, 1533 bp, synthetic construct
  source 1..1533: mol_type = other DNA; note = nucleotide sequence coding gene TAL
atgactcaagttgttgaaagacaagctgacagattgtcttctagagaatacttggctagagttgttagatctgctggttgggacgctggtttgacttcttgtactgacgaagaaatcgttagaatgggtgcttctgctagaactatcgaagaatacttgaagtctgacaagccaatctacggtttgactcaaggtttcggtccattggttttgttcgacgctgactctgaattggaacaaggcggcagcttgatctctcacctcggtactggtcaaggtgctccattggctccagaagtttctagattgatcttgtggttgagaatccaaaacatgagaaagggttactctgctgtttctccagttttctggcaaaagttggctgacctctggaacaagggcttcactccagctataccaagacacggtactgtttctgcttctggtgacttgcaaccattggctcacgctgctttggctttcactggtgttggtgaagcttggactagagacgctgacggtagatggtctactgttccagctgttgacgctttggctgctttgggtgctgaaccattcgactggccagttagagaagctttggctttcgttaacggtactggtgcttctttggctgttgctgttttgaaccacagatctgctttgagattggttagagcttgtgctgttttgtctgctagactcgcgacattgttgggtgctaatccagaacactacgacgttggtcacggtgttgctagaggtcaagttggtcaattgactgctgctgaatggatcagacaaggtttgccaagaggtatggttagagacggttctagaccattgcaagaaccatactctttgagatgtgctccacaagttttgggtgctgttttggaccaattggacggtgctggtgacgttttggctagagaagttgacggttgtcaagacaacccaatcacttacgaaggtgaattgttgcacggtggtaacttccacgctatgccagttggtttcgcttctgaccaaatcggtttggctatgcacatggctgcttacttggctgaaagacaattgggtttgttggtttctccagttactaacggtgacttgccaccaatgttgactccaagagctggtagaggtgctggtttggctggtgttcaaatctctgctacttctttcgtttctagaatcagacaattggttttcccagcttctttgactactttgccaactaacggttggaaccaagaccacgttccaatggctttgaacggtgctaactctgttttcgaagctttggaattgggttggttgactgttggttctttggctgttggtgttgctcaattggctgctatgactggtcacgctgctgaaggtgtttgggctgaattggctggtatctgtccaccattggacgctgacagaccattgggtgctgaagttagagctgctagagacttgttgtctgctcacgctgaccaattgttggttgacgaagctgacggtaaggacttcggttaa

SEQ ID NO: 29 - DNA, 398 bp, synthetic construct
  source 1..398: mol_type = other DNA; note = nucleotide sequence of promoter HXT7p
actacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 30 - DNA, 499 bp, synthetic construct
  source 1..499: mol_type = other DNA; note = nucleotide sequence of terminator PRS28At
gaagcttatttgcagattgaaggttgcagctatacaaaatctatcttatatatttgtgccttttgtttcttatatatgtcttctttaaatagtacatcactataatacaataatactaataactctattgtaactcgtgatatattcactaattatttaggtgcgaaaaaccatacccgcattgatatactttttgggtatattgagttcttccgcgttagttgttatgggccttctatggtagacaagaattttatatacgtaggcctaacattagtggctccagctgcttaccaaatccatgaccgatgagaaacgagcttttccaaaaggtaacaaccatataagatcagaaactttcaatggttcggtatctcacaaaatctccgaatcgattaaagatatcgctagcttaagaccacacggtaaatatacggtacaagacatcgacaacatcatagcatccacatcttcacatgaaaatagaggccaatcaggc

SEQ ID NO: 31 - DNA, 1350 bp, synthetic construct
  source 1..1350: mol_type = other DNA; note = nucleotide sequence of coding gene sahH of SAH hydrolase
atgtctgctccagctcaaaactacaaaatcgctgatatctctttggctgccttcggtagaaaggaaatcgaattggctgaacatgaaatgccaggtttgatggccatcagaaaggcttacggtgacgtccaacctttgaaaggcgcccgtattgctggttgtttgcacatgaccattcaaactgctgttttaattgaaactttagttgctttgggtgccgaagttacctggtcctcttgtaacatctattcgactcaagatcatgccgccgctgctattgccgcttccggtgttccagtttttgcctggaagggtgaaactgaagaagagtatttgtggtgtattgaacaacaattgtttgccttcaaggacaacaagaaattgaacttgatcttagatgatggtggtgatttaaccactttagttcatgaaaagcaccctgaaatgctggaagactgctttggtctttccgaagaaactaccaccggtgttcaccacttatacagaatggtcaaagaaggcaagttaaaggttcctgccattaacgttaacgactccgtcactaagtccaagtttgacaacttgtacggctgtagagaatccttagtcgacggtattaagagagccactgatgtcatgttggctggtaaggttgccgttgttgctggttacggtgatgtcggtaagggttgtgctgctgccttaagaggaatgggtgctcgtgtcttggttaccgaaattgacccaatcaacgctttacaagctgccatggaaggctaccaagttgttaccatggaagatgcatcccacattggtcaagttttcgttaccaccactggttgtagagatattatcaacggtgaacatttcatcaacatgccagaagatgccattgtttgtaacattggccatttcgatatcgaaattgatgtcgcctggttaaaggctaacgctaaagaatgtattaacatcaaaccacaagtcgaccgttacttgttgtcttctggtagacacgtcatcttgttggctaacggtagattagttaacttgggttgtgctactggtcactcatctttcgttatgtcttgttccttctctaaccaagtcttagctcaaattgctttgttcaagtctaacgataagtctttcagagaaaagcacattgaattccaaaagacaggcccattcgaagttggtgtccacgttttgccaaagatcttggatgaagctgtcgctaagttccacttgggcaacttgggtgttagattgactaaattgagtaaagtccaatctgaatacttgggtattccagaagaaggtccattcaaggccgaccactacagatattga

SEQ ID NO: 32 - DNA, 1182 bp, synthetic construct
  source 1..1182: mol_type = other DNA; note = nucleotide sequence of gene SAM2
atggaaactttcttgttcacttctgaatctgttaacgaaggtcacccagacaagttgtgtgaccaaatctctgacgctgttttggacgcttgtttggaacaagacccagactctaaggttgcttgtgaaacttgtactaagactaacatggttatggttttcggtgaaatcactactaaggctactatcgactacgaaaagatcgttagagacacttgtagatctatcggtttcatctctgacgacgttggtttggacgctgacaagtgtaaggttttggttaacatcgaacaacaatctccagacatcgctcaaggtgttcacggtcacttcactaagagaccagaagacatcggtgctggtgaccaaggtcacatgttcggttacgctactgacgaaactccagaattgatgccattgtctcacgttttggctactaagatcggtgctagattgactgaagttagaaagaacggtacttgtagatggttgagaccagacggtaagactcaagttactgttgaatactacaacgacaacggtgctatggttccagttagagttcacactgttttgatctctactcaacacgacgaaactgttactaacgacgaaatcgctagagacttgaaggaacacgttatcaagccaatcatcccagaaaagtacttggacgacaagactatcttccacttgaacccatctggtagattcgttatcggtggtccacacggcgacgctggtctcactggtagaaagatcatcatcgacacttacggtggttggggtgctcacggtggtggtgctttctctggtaaggacccaactaaggttgacagatctggtgcttacatcgttagacaagctgctaagtctgttgttgctaacggtatggctagaagagctttggttcaagtttcttacgctatcggtgttccagaaccattgtctgttttcgttgacacttacggtactggtttgatcccagacaaggaaatcttgaagatcgttaaggaaactttcgacttcagaccaggtatgatgactatcaacttggacttgaagagaggtggtaacggtagattccaaaagactgctgcttacggtcacttcggtagagacgacccagacttcacttgggaagttgttaagccattgaagtgggacaagccacaagcttaa

SEQ ID NO: 33 - DNA, 500 bp, synthetic construct
  source 1..500: mol_type = other DNA; note = nucleotide sequence of promoter CYC1p
aatttttttggaaaaccaagaaatgaattatatttccgtgtgagacgacatcgtcgaatatgattcagggtaacagtattgatgtaatcaatttcctacctgaatctaaaattcccgggagcaagatcaagatgttttcaccgatctttccggtctctttggccggggtttacggacgatggcagaagaccaaagcgccagttcatttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaata

SEQ ID NO: 34 - DNA, 433 bp, synthetic construct
  source 1..433: mol_type = other DNA; note = nucleotide sequence of terminator ALY2t
tctagcacaaggttcctacgtcatcccttcctgttcttacatctggcgtccagagcaaacataaaatattagaaactctaattgttatacatatatatgtttatatctgtttatggtacgttcttttctttaattttgctttttgaaaagtgatgttcgcaatctatctcactaattggttaagaacagtagttagttcgtcaggggtatatttgatatgctttctaggattcttgaaagagtctttatatctactgtagaatctttcatacatgggcataaccaacgatattatttctgacttcaaagttactttcaatgatggatctgaaagcttatactgttttgtttttgatactaaatcttcgaatccttcattgaattttctaaatttctcctttatttgttctttgtcttttgacttcttcccgct

SEQ ID NO: 35 - DNA, 402 bp, synthetic construct
  source 1..402: mol_type = other DNA; note = nucleotide sequence of terminator SCW4t
gtctactcaattgtaaaaaaaaacaaaaaaaatcttgatttaaaaaacgatacccaatatttttaactttaaattaaggctttgataatcttctttttctctgttatatcaaaggactgctttctggtttgccttcgctaatctttttttttttttttcattttcctattttagttcgtttatgatctcatttgtatttctgtatatttgtcttaatacgaaatacgagtttttattttatattctacctttcttttttttttggctaaaagcgtgtacggttctatgtaaatatgtacgattacatgtgtaatgtttgtttggccgggctcttcatctatgccgttgccttcttgcaggcggagtcctggatctgttacgatggtttctcgatcctcttgg

SEQ ID NO: 36 - DNA, 699 bp, synthetic construct
  source 1..699: mol_type = other DNA; note = nucleotide sequence of mtn
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactttgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgttgtcgtacgcgccatctccgacgtggccgatcaacagtctcatcttagcttcgatgagttcctggctgttgccgctaaacagtccagcctgatggttgagtcactggtgcagaaacttgcacatggctaa

SEQ ID NO: 37 - DNA, 516 bp, synthetic construct
  source 1..516: mol_type = other DNA; note = nucleotide sequence of luxS
atgccgttgttagatagcttcacagtcgatcatacccggatggaagcgcctgcagttcgggtggcgaaaacaatgaacaccccgcatggcgacgcaatcaccgtgttcgatctgcgcttctgcgtgccgaacaaagaagtgatgccagaaagagggatccataccctggagcacctgtttgctggttttatgcgtaaccatcttaacggtaatggtgtagagattatcgatatctcgccaatgggctgccgcaccggtttttatatgagtctgattggtacgccagatgagcagcgtgttgctgatgcctggaaagcggcaatggaagacgtgctgaaagtgcaggatcagaatcagatcccggaactgaacgtctaccagtgtggcacttaccagatgcactcgttgcaggaagcgcaggatattgcgcgtagcattctggaacgtgacgtacgcatcaacagcaacgaagaactggcactgccgaaagagaagttgcaggaactgcacatctag

SEQ ID NO: 38 - DNA, 423 bp, synthetic construct
  source 1..423: mol_type = other DNA; note = nucleotide sequence of terminator NAT5t
atttcttaacagatggctgaaaaattttttttttttttttggttttgctttgtatacacatgtatctatttttataaagatgaaatatatacgtctaagagctaaaatgaaaaactatactaatcacttatatctattctggtgattcatcatcttcagcttctgtatccgtatcgatatcagtataatcgctagcatcactttcatcatcactttcggtttcgtcgtcatcagtagagtactcatcatcctcctcagaatacacctctacttgttccactgcaggagaatcacgaactttcatattcatggtaatatccaaatctgtagtgaaataatctgtggatttaacgattactctaaaaaagaaatcaccaacagtttcaggtgcaggctgacctaatggaatttttatggtcccga

SEQ ID NO: 39 - DNA, 462 bp, synthetic construct
  source 1..462: mol_type = other DNA; note = nucleotide sequence of terminator IDP1t
tcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcaatttccaacattttcatgagttcttcttcttcattaccgtgttttagggggctgttcgcacttctaatagggctatcaccaagctgttctaattcgtccaaaagttcagtaacacgatctttatgctttagctcgtcataatctttcaattcataaatatttacaatttcgtctacgatattaaattgcctcttgtaggtgcctatcttttccttatgctcttcattttcaccgttttcttgaaaccaaacaccgaactcactacgca

SEQ ID NO: 40 - DNA, 1000 bp, synthetic construct
  source 1..1000: mol_type = other DNA; note = nucleotide sequence of promoter module pBM16A-GPM1p
aatgctactattttggagattaatctcagtacaaaacaatattaaaaagaggtgaattatttttccccccttattttttttttgttaaaattgatccaaatgtaaataaacaatcacaaggaaaaaaaaaaaaaaaaaaaaaatagccgccatgaccccggatcgtcggttgtgatacggtcagggtagcgccctggtcaaacttcagaactaaaaaaataataaggaagaaaaaaatagctaatttttccggcagaaagattttcgctacccgaaagtttttccggcaagctaaatggaaaaaggaaagattattgaaagagaaagaaagaaaaaaaaaaaatgtacacccagacatcgggcttccacaatttcggctctattgttttccatctctcgcaacggcgggattcctctatggcgtgtgatgtctgtatctgttacttaatccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaaatgcatattttttctcttggtaaatgaatgcttgtgatgtcttccaagtgatttcctttccttcccatatgatgctaggtacctttagtgtcttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcaca

SEQ ID NO: 41 - DNA, 500 bp, synthetic construct
  source 1..500: mol_type = other DNA; note = nucleotide sequence of ASP3t
agcctgatcagtgacttttcatcacactgtgtttttatatagttcttagtagtaaatatactctataatttctacatccttttcatgatgtgccagttaagctatttttagcaaacaaaaagatgaatttgagtattgagcacatcattcactgttcagctgatacaaaacatatgatttctaatattcacataggaaaaatcgctccaaacatcgaagtatcatcgctactttggtcttttatctgcttctcttctcttcgctcttttcactgctcgtgccgctgcaacgctccaactcagttctatctcagctccttccccttatcccttgagcagtgtcaaggcgaggctgcgtgatgtattcgtgcgcgaaaaaaaaaacaactgcagcacctgagtttcgcgtatggtcacccactacactactcggtcaggctcttaccagcttaactacagttgatcggacgggaaacggtgctttctggtagatatggccgc

SEQ ID NO: 42 - DNA, 492 bp, synthetic construct
  source 1..492: mol_type = other DNA; note = nucleotide sequence MDM35t
tttagcacagaatgtgcattattcaacatgtaaatactaatactgcaatatcgacttataataatgtatagtgatccgtatattaatagatctgtttcaattctttacctttttaggatatccgtcacccgtgattccgtcggaggtgagcactcgcccaaataaataacgggaaatggtggcaaaaagtagtggcgggaaaaggaaaaattttcgttctctcccatataaacgtttcattccttttcctaagtcttttacagtaatttcagaaacattcgtattttatatttgatcttttgaagctacaagaaaaactcttaccaattaccccaaaaaaatcaccatcataaagtacttacatatttatttttgtttggtcgttttctcaatataatctacatcatcatatatatataatgtctacacaatttaggaagtctaatcataatagtcatagtagtaaaaaactaaatcctgcgctaaagtcca

SEQ ID NO: 43 - DNA, 711 bp, synthetic construct
  source 1..711: mol_type = other DNA; note = nucleotide sequence of delta 22site1 plasmid
caccggagcttggatatgataaacgaaatattcttgaatcgtgagatcgcctgttttcaaaaccgttggaggcagaaacaattttgtcacaagatgggcattctaccccatccttgctgtattattgtagtctcgctttcttttatgctggacaaatgagactactgcacatttttatacgttcttggttttttttaaaggtgtggtttcggcattatcctgccgcacgtttcttggataattcatcctgattctctattttaaacgcttcagcctatcaggatttggttttgatacatactgcaagagtgtatctcgggaacagtcatttattccgcaacaaacttaattgcggaacgcgttaggcgatttctagcatatatcaaataccgttcgcgatttcttctgggttcgtctcttttcttttaaatacttattaacgtactcaaacaactacacttcgttgtatctcagaatgagatccctcagtatgacaatacatcattctaaacgttcgtaaaacacatatgaaacaactttataacaaagcgaacaaaatgggcaacatgagatgaaactccgcgtcccttagctgaactacccaaacgtacgaatgcctgaacaattagtttagatccgagattccgcgcttccatcatttagtataatccatattttatataatatataggataagtaacagcccgcgaa

SEQ ID NO: 44 - DNA, 653 bp, synthetic construct
  source 1..653: mol_type = other DNA; note = nucleotide sequence of delta 22site2 plasmid
ggaccaactatcatccgctaattactgacattaccaaatgagatctgtgaatgggcaagataaaaaacaaaaattgaaatgtttgacgttatgtaaaactattaattccttcgctttcggcggtcacagaatttgcgtgtagctgactcttgttcaatcaatatcatttgttactttatttgaaagtctgtattactgcgcctattgtcatccgtaccaaagaacgtcaaaaagaaacaagataatttttgtgcttacaccatttatagatcactgagcccagaatatcgctggagctcagtgtaagtggcatgaacacaactctgactgatcgcacatattgccgttatcataaatactagttgtacttgtcaatgcgacgaatggcatcatgcctattattacgttcctctttttccgtttcatgtttccagaatgctattgaatctaacacttcaattataaaaaagaataaatccgcaataattttaggctaattgttgtactgtcaagcgaacctaatggttaaaattcagaggaaccttcgacgtagtctgatcgctacttctatatcttatgttcccagtcaatcaaaagttgatactataatagctgccatttatacctgttagttatggcgatcgtttatcacg

SEQ ID NO: 45 - DNA, 1467 bp, synthetic construct
  source 1..1467: mol_type = other DNA; note = nucleotide sequence of functional gene Kas
atgtcttctatcacttcttcttgttactctaacttgatcttgaagaagaaggaatctagaatcaacggtggtttccacttgcaatacaacggtttgagagctatcgaatctgttcaaatgccagcttcttctgttgcttacaagccaaaggttgttaagtggggtagaatcagagttatggcttctccagctgtttctgctccaaagagagaaactgacccaaagaagagaatcgttatcactggtatgggtttggtttctgttttcggttctgacatcgacaacttctacaacaagttgttggaaggtcaatctggtatctctttgatcgacagattcgacgcttcttcttacactgttagattcgctggtcaaatcagagacttctcttctgaaggttacatcgacggtaagaacgacagaagattggacgactgttggagatactgtttggttgctggtaagagagctttggaagacgctaacctcggtcagcaagtactagacactatggacaagactagaatcggtgttttggttggttcttctatgggtggttctaaggttttcgctgacgctgttgaagctttggttcaaagaggttacaagaagatcaacccattcttcatcccatactctatcactaacatgggttctgctttgttggctatcgacactggtttgatgggtccaacttactctatctctactgcttgtgctactgctaactactgtttctacgcttctgctaaccacatcagaagaggtgaagctgacatcatggttgctggtggtactgacgctttcatctctgctatcggtgttggtggtttgatcgcttgccgtgcgttgtctcaaagaaatgacgaatacgaaaaggcttctagaccatgggacagaaacagagacggtttcgttatcggtgaaggttctggtgttttggttatggaaaacttggaacacgctttgaagagaggtgctccaatcatcgctgaatacttgggtggtgctatcacttgtgacgctcaccacatcactgacccaagagctgacggtttgggtgtttcttcttgtatcgttatgtctttggttgacgctggtgtttctccagaagaagttaactacatcaacgctcacgctacttctactttggctggtgacttggctgaagttaacgctatcaagaaggttttcaaggacacttctgaaatcaagatgaacggtactaagtctatgatcggtcacggtttgggtgcttctggtggtttggaagctatcgctactatcaaggctatcactactggttggttgcacccaactatcaaccaatacgacttggaaccacaagttactatcgacactgttccaaacgttaagaagcaacacgaagttaacgttggtatctctaactctttcggtttcggtggtcacaattctgttgttgtcttcgccccatacaagccataa

SEQ ID NO: 46 - DNA, 399 bp, synthetic construct
  source 1..399: mol_type = other DNA; note = nucleotide sequence of functional gene Acl
atggcttctatcactgcttcttctttcgctatctcttctttcaagcacaaccaaatctgtttgaagtcgtcgagcttgccaatcaagggcatctctttcccatctttgagaatgaagccagctactagaagattgtctatcaactgtgctgctaagccagaaactgttgacaaggtttgtgctatcgttaagaagcaattggctttgccaggtgactctgttgttaacggtgaatctaagtttgctgctctcggtgctgactcgttggacactgttgaaatcgttatgggtttggaagaagctttcggtatcactgttgaagaagaaaacgctcaaactatcgctactgttcaagacgctgctgacttgatcgaagacttggttgctaagaagtgttaa

SEQ ID NO: 47 - DNA, 1116 bp, synthetic construct
  source 1..1116: mol_type = other DNA; note = nucleotide sequence of functional gene Fat
atgttgtctagaggttctttcttgaagaacgttgctatcggtgaccacaaccaatgtactaagagattcttcaactctaactctgtttctatcggttgtagaaagccagttatctcttcttcttcttcttctgttggtgttagatctggtccagttttggctgttgctactaacgaaagagaatctaagtctaagcaacaagtttctcacgaaccatctttggctgacagattgagattgggtactatgtctgaagacggtatgtcttacaaggaaaagttcatcgttagatgttacgaagttggtgttaacaagactttgactgttgaaactatcgctaacttgttgcaagaagttggttgtaaccacgctcaatctgttggtttctctactgacggtttcgctactactcactctatgagaaagttgcacttgatctgggttactgctagaatgtacatcgaaatctacaagtacccagcttggtctgacgttgttgaaatcgaaacttggtgtcaatctgaaggtagaatcggtactagaagagactggatcttgaaggactgtgctactggtgaagttatcggtagagctacttctaagtggttgatgatgaaccaagacactagaagattgcaaaaggttactgacgaagttagagacgaattggaattgtacttcccaaaggaattgagattggctttcccagaagaaaacaacggttctttgaagaagatcccaaagttggaagacccagctgaatactctaagctcggcttggttccaagacgtgctgacttggacatgaaccaacacgttaacaacgttacttacatcggttgggttttggaatctatgccacaagaaatcatcgacactcacgaattgggtactatcactatcgactacagaagagaatgtcaacacgacgacgttgttgacagcttgacttctgttgagccaatcgaagacactgacgctttgggtgctaacggttctgctactgctgctaaggacgttaacaagtctgttttgcacttcttgagattgtcttctgacggtttggaaatcaacagatgtagaactgaatggagaaagaagccatctagaatctaa

SEQ ID NO: 48 - DNA, 1323 bp, synthetic construct
  source 1..1323: mol_type = other DNA; note = nucleotide sequence of functional gene AT3
atggctttcgctttgccatcttctttggtttctgtttgtgacaagtctttcatcaagccatcttctttgactccatctactttgagattccacaagttgtctttcatcgaccaatctttgtctaacatgtacatcccatgcgcgttcttctacccaaaggttcaacaaagattggaagactctaagaactctgacgaattgtctcacatcgctcacttgttgcaaacttctttgtctcaaactttggtttcttactacccatacgctggtaagttgaaggacaacgctactgttgactgtaacgacatgggtgctgaattcttgtctgttagaatcaagtgttctatgtctgaaatcttggaccacccacacgcttctttggctgaatctatcgttttgccaaaggacttgccatgggctaacaactgtgaaggtggtaacttgttggttgttcaagtttctaagttcgactgtggtggtatcgctatctctgtttgtttctctcacaagatcggtgacggttgttctttgttgaacttcttgaacgactggtcttctgttactagagacccaactactactactttggttccatctccaagattcgttggtgactctgttttctctactcaaaagtacggttctttgatcactccacaaatcttgtctgacttgaaccaatgtgttcaaaagagattgatcttcccaactgacaagctcgacgctctcagagctaaggttgctgaagaatctggtgttaagaacccaactagagctgaagttgttagtgcgcttttgtttaaatgtgctactaaggcttcttcttctatgttgccatctaagttggttcacttcttgaacatcagaactatgatcaagccaagattgccaagaaacgctatcggtaacttgtcttctatcttctctatcgaagctactaacatgcaagacatggaattgccaactttggttagaaacttgagaaaggaagttgaagttgcttacaagaaggaccaagttgaacaaaacgaattgatcttggaagttgttgaatctatgagagaaggtaagttgccattcgaaaacatggacggttacaagaacgtttacacttgttctaacttgtgtaagtacccatactacactgttgacttcggttggggtagaccagaaagagtttgtttgggtaacggtccatctaagaacgctttcttcttgaaggactacaaggctggtcaaggtgttgaagctagagttatgttgcacaagcaacaaatgtctgaattcgaaagaaacgaagaattggttgaattcatcgcttaa

SEQ ID NO: 49 - DNA, 437 bp, synthetic construct
  source 1..437: mol_type = other DNA; note = nucleotide sequence of HAP4t
aataggcatgttgcaataaaacgaaaacaactaaaaatcacgaaaacaaaatgatattatacaataaaaaattcttattatgggtaatgatagtattcttcgcctgcttaggcgtccttttccttcaacaacaaaaattccaaaaaaaaaaagtaaaaaaacaaaactttgattgttttttaatgatgttaatgattttttttttctttctttatcataaaaaaaaagttaaaatgaaaaacaaatatgggtctggaaggccattattttttttttatttatataccgtttctggtacttagttatttattctcatacatacactatattcaaattacctaagagcattttcacatatccgtttactttcattttttttttttttgcttcctttttacatatcttccgtatatcacatcacgtttacgcgtatggtg

SEQ ID NO: 50 - DNA, 434 bp, synthetic construct
  source 1..434: mol_type = other DNA; note = nucleoside sequence of YCP4t
gaaacaacagttcgcgtctaatatttagttcttactaacattaattctatctcagcagatatatcaatgtaggacgttggaaaattaataaagaattaactctttttattttattttttcctttttatattaaaagtatatttctgtaagtccttgtactaataatatcatttcctaatattttgaaatatagttgttttttgtcaagcaaaactcttgatttcaaagacctcattttgttaagccaaaagcgaaaaattctatttcgacttttctttctgtttccgctccttgtattctaccattgatgctgtcttaaacaataaatatacagtttgaaattaagtttctatatacaggcttaagtttatttaaaaaggaaaccactagatgaaagatgagcctagtccttttttaaagtccgaacgcgacgt

SEQ ID NO: 51 - DNA, 35 bp, synthetic construct
  source 1..35: mol_type = other DNA; note = Primer 1
cctgctcttcacattttgtttatgtgtgtttattc

SEQ ID NO: 52 - DNA, 59 bp, synthetic construct
  source 1..59: mol_type = other DNA; note = Primer 2
tctgttctattgtatatctcccctccgccacctacatgtatactagcgttgaatgttag

SEQ ID NO: 53 - DNA, 59 bp, synthetic construct
  source 1..59: mol_type = other DNA; note = Primer 3
aaacttcttgttgttgacgctaacattcaacgctagtatacatgtaggtggcggagggg

SEQ ID NO: 54 - DNA, 36 bp, synthetic construct
  source 1..36: mol_type = other DNA; note = Primer 4
cctgctcttcatgtatatgagatagttgattgtatg

SEQ ID NO: 55 - DNA, 37 bp, synthetic construct
  source 1..37: mol_type = other DNA; note = Primer 5
cctgctcttcacattgttttatatttgttgtaaaaag

SEQ ID NO: 56 - DNA, 59 bp, synthetic construct
  source 1..59: mol_type = other DNA; note = Primer 6
ggaagcgcctacgcttgacatctactatatgtaagtataacgcacagatattataacat

SEQ ID NO: 57 - DNA, 59 bp, synthetic construct
  source 1..59: mol_type = other DNA; note = Primer 7
gcaaatgcctattgtgcagatgttataatatctgtgcgttatacttacatatagtagat

SEQ ID NO: 58 - DNA, 39 bp, synthetic construct
  source 1..39: mol_type = other DNA; note = Primer 8
cctgctcttcatgtggtactagtgtttagttaattatag

SEQ ID NO: 59 - DNA, 42 bp, synthetic construct
  source 1..42: mol_type = other DNA; note = Primer 9
cctgctcttcaggtttaacaaattggaataggagcaccgttc

SEQ ID NO: 60 - DNA, 38 bp, synthetic construct
  source 1..38: mol_type = other DNA; note = Primer 10
cctgctcttcagtaatggaccaaattgaggcaatgttg

SEQ ID NO: 61 - DNA, 38 bp, synthetic construct
  source 1..38: mol_type = other DNA; note = Primer 11
cctgctcttcaacaatggacttgttgttgttggaaaag

SEQ ID NO: 62 - DNA, 38 bp, synthetic construct
  source 1..38: mol_type = other DNA; note = Primer 12
cctgctcttcagatttaacaatttcttggcttcataac

SEQ ID NO: 63 - DNA, 38 bp, synthetic construct
  source 1..38: mol_type = other DNA; note = Primer 13
cctgctcttcaggtttaacataacttagcttttaaatc

SEQ ID NO: 64 - DNA, 36 bp, synthetic construct
  source 1..36: mol_type = other DNA; note = Primer 14
cctgctcttcagtaatgattactgcagcattgcacg

SEQ ID NO: 65 - DNA, 37 bp, synthetic construct
  source 1..37: mol_type = other DNA; note = Primer 15
cctgctcttcaacaatgaaaattgaagttaaagaatc

SEQ ID NO: 66 - DNA, 42 bp, synthetic construct
  source 1..42: mol_type = other DNA; note = Primer 16
cctgctcttcagatttaaaaatcgtacaaaaatttctcaaac

SEQ ID NO: 67 - DNA, 37 bp, synthetic construct
  source 1..37: mol_type = other DNA; note = Primer 17
cctgctcttcacatggtctcacatctattattgtatg

SEQ ID NO: 68 - DNA, 59 bp, synthetic construct
  source 1..59: mol_type = other DNA; note = Primer 18
ttgtacatactaatatatataaacaaagtaacgtctcctacgcggcgttatgtcactaa

SEQ ID NO: 69 - DNA, 59 bp, synthetic construct
  source 1..59: mol_type = other DNA; note = Primer 19
cgcaagttggtgcacgtcgttagtgacataacgccgcgtaggagacgttactttgttta

SEQ ID NO: 70 - DNA, 36 bp, synthetic construct
  source 1..36: mol_type = other DNA; note = Primer 20
cctgctcttcatgtataatttgcgatcgtagttggg

SEQ ID NO: 71 - DNA, 37 bp, synthetic construct
  source 1..37: mol_type = other DNA; note = Primer 21
cctgctcttcaggtttaagatattcttctgcacaaag

SEQ ID NO: 72 - DNA, 39 bp, synthetic construct
  source 1..39: mol_type = other DNA; note = Primer 22
cctgctcttcagtaatggctactaacggtagacaccaag

SEQ ID NO: 73 - DNA, 36 bp, synthetic construct
  source 1..36: mol_type = other DNA; note = Primer 23
cctgctcttcaacaatgtctgatgaattgacatttg

SEQ ID NO: 74 - DNA, 37 bp, synthetic construct
  source 1..37: mol_type = other DNA; note = Primer 24
cctgctcttcagatttaatcttctcttctataaccac

SEQ ID NO: 75 - DNA, 59 bp, synthetic construct
  source 1..59: mol_type = other DNA; note = Primer 25
cttgtattgttctaacgatccgaccatttcatcctgaatataacaatactgacagtact

SEQ ID NO: 76 - DNA, 59 bp, synthetic construct
  source 1..59: mol_type = other DNA; note = Primer 26
catatcatgacccataaattcgtttgtaatatttgccattttgaatatgtattacttgg

SEQ ID NO: 77 - DNA, 59 bp, synthetic construct
  source 1..59: mol_type = other DNA; note = Primer 27
ttgtcatatataaccataaccaagtaatacatattcaaaatggcaaatattacaaacga

SEQ ID NO: 78 - DNA, 59 bp, synthetic construct
  source 1..59: mol_type = other DNA; note = Primer 28
cgagtaaaaaaagagctgcagttcccaggaagcgttaaattattgtttttgtgacttca

SEQ ID NO: 79 - DNA, 59 bp, synthetic construct
  source 1..59: mol_type = other DNA; note = Primer 29
gaaaaaagagttgaagaattgaagtcacaaaaacaataatttaacgcttcctgggaact

SEQ ID NO: 80 - DNA, 59 bp, synthetic construct
  source 1..59: mol_type = other DNA; note = Primer 30
gtgatgaattttgagagcccacttttgttggggacgattgagaggctgaaggcagagaa

SEQ ID NO: 81 - DNA, 59 bp, synthetic construct
  source 1..59: mol_type = other DNA; note = Primer 31
gcatccacatcttcacatgaaaatagaggccaatcaggcaggagacgttactttgttta

SEQ ID NO: 82 - DNA, 59 bp, synthetic construct
  source 1..59: mol_type = other DNA; note = Primer 32
tttctctcgcagcgccagtaacgggttttccgatgtcatataatttgcgatcgtagttg

SEQ ID NO: 83 - DNA, 59 bp, synthetic construct
  source 1..59: mol_type = other DNA; note = Primer 33
tacatacacaacataaacccaactacgatcgcaaattatatgacatcggaaaacccgtt

SEQ ID NO: 84 - DNA, 59 bp, synthetic construct
  source 1..59: mol_type = other DNA; note = Primer 34
ctgataataaaaacggtatgcctacacatacacgctttatcaggttggatcaacaggca

SEQ ID NO: 85 - DNA, 59 bp, synthetic construct
  source 1..59: mol_type = other DNA; note = Primer 35
tacccaagtgagaacgtagtgcctgttgatccaacctgataaagcgtgtatgtgtaggc

SEQ ID NO: 86 - DNA, 59 bp, synthetic construct
  source 1..59: mol_type = other DNA; note = Primer 36
attatattatatgggtctgcaaggtagaggcgcgcttgtgcagcgatcagcaataatga

SEQ ID NO: 87 - DNA, 59 bp, synthetic construct
  source 1..59: mol_type = other DNA; note = Primer 37
attccgcgtttgcttcatttcattattgctgatcgctgcacaagcgcgcctctaccttg

SEQ ID NO: 88 - DNA, 59 bp, synthetic construct
  source 1..59: mol_type = other DNA; note = Primer 38
ggaagtgtaatcaacagggggggcagaaccagcagtcattgttaattcagtaaattttc

SEQ ID NO: 89 - DNA, 59 bp, synthetic construct
  source 1..59: mol_type = other DNA; note = Primer 39
cttttttcttcccaagatcgaaaatttactgaattaacaatgactgctggttctgcccc

SEQ ID NO: 90 - DNA, 59 bp, synthetic construct
  source 1..59: mol_type = other DNA; note = Primer 40
cctctcctagcgtacacggatggcaagaataaactggcttcaacttttatagttgtcaa

SEQ ID NO: 91 - DNA, 59 bp, synthetic construct
  source 1..59: mol_type = other DNA; note = Primer 41
agtggcataaaagaattttttgacaactataaaagttgaagccagtttattcttgccat

SEQ ID NO: 92 - DNA, 58 bp, synthetic construct
  source 1..58: mol_type = other DNA; note = Primer 42
agctgaaatgcaaggattgataaaataataggataatagaagaatggatcaaaatggc

SEQ ID NO: 93 - DNA, 59 bp, synthetic construct
  source 1..59: mol_type = other DNA; note = Primer 43
ataacatcttttttttattccttctttgatattttgtcaaactgtgggaatactcaggt

SEQ ID NO: 94 - DNA, 59 bp, synthetic construct
  source 1..59: mol_type = other DNA; note = Primer 44
aacacgcaggggcccgaaattgttcctacgagaagtagttgcttacctgtattccttta

SEQ ID NO: 95 - DNA, 59 bp, synthetic construct
  source 1..59: mol_type = other DNA; note = Primer 45
aggagaaaaaggaggatagtaaaggaatacaggtaagcaactacttctcgtaggaacaa

SEQ ID NO: 96 - DNA, 59 bp, synthetic construct
  source 1..59: mol_type = other DNA; note = Primer 46
agacaatctgtcagcttgtctttcaacaacttgagtcattttttaattttaatcaaaaa

SEQ ID NO: 97 - DNA, 59 bp, synthetic construct
  source 1..59: mol_type = other DNA; note = Primer 47
aacacaaaaacaaaaagtttttttaattttaatcaaaaaatgactcaagttgttgaaag

SEQ ID NO: 98 - DNA, 59 bp, synthetic construct
  source 1..59: mol_type = other DNA; note = Primer 48
ttttgtatagctgcaaccttcaatctgcaaataagcttcttaaccgaagtccttaccgt

SEQ ID NO: 99 - DNA, 60 bp, synthetic construct
  source 1..60: mol_type = other DNA; note = Primer 49
tttgttggttgacgaagctgacggtaaggacttcggttaagaagcttatttgcagattga

SEQ ID NO: 100 - DNA, 59 bp, synthetic construct
  source 1..59: mol_type = other DNA; note = Primer 50
ttgtacatactaatatatataaacaaagtaacgtctcctgcctgattggcctctatttt

SEQ ID NO: 101 - DNA, 59 bp, synthetic construct
  source 1..59: mol_type = other DNA; note = Primer 51
tatagacacacaaacacaaatacacacactaaattaataatgtctgctccagctcaaaa

SEQ ID NO: 102 - DNA, 59 bp, synthetic construct
  source 1..59: mol_type = other DNA; note = Primer 52
taagaacaggaagggatgacgtaggaaccttgtgctagatcaatatctgtagtggtcgg

SEQ ID NO: 103 - DNA, 59 bp, synthetic construct
  source 1..59: mol_type = other DNA; note = Primer 53
cataacaccaagcaactaatactataacatacaataataatggaaactttcttgttcac

SEQ ID NO: 104 - DNA, 59 bp, synthetic construct
  source 1..59: mol_type = other DNA; note = Primer 54
atcaagattttttttgtttttttttacaattgagtagacttaagcttgtggcttgtccc

SEQ ID NO: 105 - DNA, 59 bp, synthetic construct
  source 1..59: mol_type = other DNA; note = Primer 55
accctgccctcatatcacctgcgtttccgttaaactatcctagtacactctatattttt

SEQ ID NO: 106 - DNA, 59 bp, synthetic construct
  source 1..59: mol_type = other DNA; note = Primer 56
gccaagtaggcaattatttagtactgtcagtattgttatcctgatgcggtattttctcc

SEQ ID NO: 107 - DNA, 59 bp, synthetic construct
  source 1..59: mol_type = other DNA; note = Primer 57
ataccgcacagatgcgtaaggagaaaataccgcatcaggaatttttttggaaaaccaag

SEQ ID NO: 108 - DNA, 59 bp, synthetic construct
  source 1..59: mol_type = other DNA; note = Primer 58
gatatcagcgattttgtagttttgagctggagcagacattattaatttagtgtgtgtat

SEQ ID NO: 109 - DNA, 59 bp, synthetic construct
  source 1..59: mol_type = other DNA; note = Primer 59
ctcctttatttgttctttgtcttttgacttcttcccgctacgcggcgttatgtcactaa

SEQ ID NO: 110 - DNA, 59 bp, synthetic construct
  source 1..59: mol_type = other DNA; note = Primer 60
ttcgttaacagattcagaagtgaacaagaaagtttccattattattgtatgttatagta

SEQ ID NO: 111 - DNA, 59 bp, synthetic construct
  source 1..59: mol_type = other DNA; note = Primer 61
gaagaaggtccattcaaggccgaccactacagatattgatctagcacaaggttcctacg

SEQ ID NO: 112 - DNA, 59 bp, synthetic construct
  source 1..59: mol_type = other DNA; note = Primer 62
cgcaagttggtgcacgtcgttagtgacataacgccgcgtagcgggaagaagtcaaaaga

SEQ ID NO: 113 - DNA, 59 bp, synthetic construct
  source 1..59: mol_type = other DNA; note = Primer 63
gttgttaagccattgaagtgggacaagccacaagcttaagtctactcaattgtaaaaaa

SEQ ID NO: 114 - DNA, 59 bp, synthetic construct
  source 1..59: mol_type = other DNA; note = Primer 64
agactgtcaaggagggtattctgggcctccatgtcgctgccaagaggatcgagaaacca

SEQ ID NO: 115 - DNA, 59 bp, synthetic construct
  source 1..59: mol_type = other DNA; note = Primer 65
ttgtcatatataaccataaccaagtaatacatattcaaaatgaaaatcggcatcattgg

SEQ ID NO: 116 - DNA, 59 bp, synthetic construct
  source 1..59: mol_type = other DNA; note = Primer 66
aaaaaaaaaaaaaaatttttcagccatctgttaagaaatttagccatgtgcaagtttct

SEQ ID NO: 117 - DNA, 59 bp, synthetic construct
  source 1..59: mol_type = other DNA; note = Primer 67
caagctataccaagcatacaatcaactatctcatatacaatgccgttgttagatagctt

SEQ ID NO: 118 - DNA, 59 bp, synthetic construct
  source 1..59: mol_type = other DNA; note = Primer 68
tgaaaaaaaaaagtggtagattgggctacgtaaattcgactagatgtgcagttcctgca

SEQ ID NO: 119 - DNA, 59 bp, synthetic construct
  source 1..59: mol_type = other DNA; note = Primer 69
ataccgcacagatgcgtaaggagaaaataccgcatcaggataacaatactgacagtact

SEQ ID NO: 120 - DNA, 59 bp, synthetic construct
  source 1..59: mol_type = other DNA; note = Primer 70
aacttcttcttccattgcaccaatgatgccgattttcattttgaatatgtattacttgg

SEQ ID NO: 121 - DNA, 59 bp, synthetic construct
  source 1..59: mol_type = other DNA; note = Primer 71
caggtgcaggctgacctaatggaatttttatggtcccgaacatgtaggtggcggagggg

SEQ ID NO: 122 - DNA, 59 bp, synthetic construct
  source 1..59: mol_type = other DNA; note = Primer 72
ccgggtatgatcgactgtgaagctatctaacaacggcattgtatatgagatagttgatt

SEQ ID NO: 123 - DNA, 59 bp, synthetic construct
  source 1..59: mol_type = other DNA; note = Primer 73
caccgttttcttgaaaccaaacaccgaactcactacgcaacgcggcgttatgtcactaa

SEQ ID NO: 124 - DNA, 59 bp, synthetic construct; identical to SEQ ID NO: 106
  source 1..59: mol_type = other DNA; note = Primer 74

SEQ ID NO: 125 - DNA, 59 bp, synthetic construct
  source 1..59: mol_type = other DNA; note = Primer 75
atggttgagtcactggtgcagaaacttgcacatggctaaatttcttaacagatggctga

SEQ ID NO: 126 - DNA, 59 bp, synthetic construct
  source 1..59: mol_type = other DNA; note = Primer 76
tctgttctattgtatatctcccctccgccacctacatgttcgggaccataaaaattcca

SEQ ID NO: 127 - DNA, 59 bp, synthetic construct
  source 1..59: mol_type = other DNA; note = Primer 77
gcactgccgaaagagaagttgcaggaactgcacatctagtcgaatttacgtagcccaat

SEQ ID NO: 128 - DNA, 59 bp, synthetic construct
  source 1..59: mol_type = other DNA; note = Primer 78
cgcaagttggtgcacgtcgttagtgacataacgccgcgttgcgtagtgagttcggtgtt

SEQ ID NO: 129 - DNA, 40 bp, synthetic construct
  source 1..40: mol_type = other DNA; note = Primer 79
cctgctcttcacattgttttatatttgttgtaaaaagtag

SEQ ID NO: 130 - DNA, 59 bp, synthetic construct
  source 1..59: mol_type = other DNA; note = Primer 80
gccaagtaggcaattatttagtactgtcagtattgttatacgcacagatattataacat

SEQ ID NO: 131 - DNA, 59 bp, synthetic construct
  source 1..59: mol_type = other DNA; note = Primer 81
gcaaatgcctattgtgcagatgttataatatctgtgcgtataacaatactgacagtact

SEQ ID NO: 132 - DNA, 39 bp, synthetic construct
  source 1..39: mol_type = other DNA; note = Primer 82
cctgctcttcatgttttgaatatgtattacttggttatg

SEQ ID NO: 133 - DNA, 38 bp, synthetic construct
  source 1..38: mol_type = other DNA; note = Primer 83
cctgctcttcacatattgtaatatgtgtgtttgtttgg

SEQ ID NO: 134 - DNA, 59 bp, synthetic construct
  source 1..59: mol_type = other DNA; note = Primer 84
attatattatatgggtctgcaaggtagaggcgcgcttgtcacatgcagtgatgcacgcg

SEQ ID NO: 135 - DNA, 59 bp, synthetic construct
  source 1..59: mol_type = other DNA; note = Primer 85
atgtaacttagcaccatcgcgcgtgcatcactgcatgtgacaagcgcgcctctaccttg

SEQ ID NO: 136 - DNA, 38 bp, synthetic construct
  source 1..38: mol_type = other DNA; note = Primer 86
cctgctcttcatgttgttaattcagtaaattttcgatc

SEQ ID NO: 137 - DNA, 38 bp, synthetic construct
  source 1..38: mol_type = other DNA; note = Primer 87
cctgctcttcaggtttatggcttgtatggggcgaagac

SEQ ID NO: 138 - DNA, 37 bp, synthetic construct
  source 1..37: mol_type = other DNA; note = Primer 88
cctgctcttcagtaatgtcttctatcacttcttcttg

SEQ ID NO: 139 - DNA, 41 bp, synthetic construct
  source 1..41: mol_type = other DNA; note = Primer 89
cctgctcttcaacaatggcttctatcactgcttcttctttc

SEQ ID NO: 140 - DNA, 41 bp, synthetic construct
  source 1..41: mol_type = other DNA; note = Primer 90
cctgctcttcagatttaacacttcttagcaaccaagtcttc

SEQ ID NO: 141 - DNA, 37 bp, synthetic construct
  source 1..37: mol_type = other DNA; note = Primer 91
cctgctcttcaggtttagattctagatggcttctttc

SEQ ID NO: 142 - DNA, 38 bp, synthetic construct
  source 1..38: mol_type = other DNA; note = Primer 92
cctgctcttcagtaatgttgtctagaggttctttcttg

SEQ ID NO: 143 - DNA, 37 bp, synthetic construct
  source 1..37: mol_type = other DNA; note = Primer 93
cctgctcttcaacaatggctttcgctttgccatcttc

SEQ ID NO: 144 - DNA, 38 bp, synthetic construct
  source 1..38: mol_type = other DNA; note = Primer 94
cctgctcttcagatttaagcgatgaattcaaccaattc

(19) United States
(12) Patent Application Publication
(10) Pub. No.: US 2024/0294929 A1
(43) Pub. Date: Sep. 5, 2024

(54) ENGINEERED SACCHAROMYCES CEREVISIAE STRAINS FOR PRODUCTION OF VANILLYLAMINE AND CAPSAICIN, AND CONSTRUCTION METHOD AND USE THEREOF

(71) Applicant: SHIHEZI UNIVERSITY, Shihezi (CN)

(72) Inventors: Genlin ZHANG, Shihezi (CN); Yuanyuan LI, Shihezi (CN); Jinyu ZHAO, Shihezi (CN); Di QIU, Shihezi (CN); Yan ZHANG, Shihezi (CN)

(21) Appl. No.: 18/522,708

(22) Filed: Nov. 29, 2023

Related U.S. Application Data
(63) Continuation of application No. PCT/CN2023/086755, filed on Apr. 7, 2023.

(30) Foreign Application Priority Data
Mar. 1, 2023 (CN) 202310194657.5

(51) Int. Cl. C12N 15/81; C12P 13/00; C12P 13/02

(52) U.S. Cl. CPC C12N 15/81; C12P 13/001; C12P 13/02

(57) ABSTRACT

The present disclosure provides engineered Saccharomyces cerevisiae strains for production of vanillylamine and capsaicin, and a construction method and use thereof, and belongs to the technical field of bioengineering. In the present disclosure, a synthesis pathway of vanillylamine is expressed heterologously in Saccharomyces cerevisiae for the first time, and biosynthesis of the vanillylamine is realized for the first time. After the optimization of precursors supply and the optimization of S-adenosyl-L-methionine (SAM) cycle, a yield of the vanillylamine reaches 16.48 g/L, and a synthesis efficiency of the vanillylamine is increased by nearly 155%. This is the highest optimization efficiency obtained by optimizing the SAM cofactor strategy so far.

### CROSS REFERENCE TO RELATED APPLICATION

The present application is a continuation application of International Patent Application No. PCT/CN2023/086755, filed on Apr. 7, 2023, which claims priority to the Chinese Patent Application No. 202310194657.5, filed with the China National Intellectual Property Administration (CNIPA) on Mar. 1, 2023, and entitled “ENGINEERING SACCHAROMYCES CEREVISIAE FOR PRODUCTION OF VANILLYLAMINE AND CAPSAICIN, AND CONSTRUCTION METHOD AND USE THEREOF”, which is incorporated herein by reference in its entirety.

### REFERENCE TO SEQUENCE LISTING

A computer readable XML file entitled “Sequence Listing”, that was created on Oct. 30, 2023, with a file size of 193,606 bytes, contains the sequence listing for this application, has been filed with this application, and is hereby incorporated by reference in its entirety.

### TECHNICAL FIELD

The present disclosure belongs to the technical field of bioengineering, and in particular relates to engineered Saccharomyces cerevisiae strains for production of vanillylamine and capsaicin, and a construction method and use thereof.

### BACKGROUND

As an indispensable bulk chemical in the chemical industry, primary benzylamine plays an important role in the production of biological compounds, personal care products, and polymers synthesis. Vanillylamine, also known as 3-methoxy-4-hydroxy benzylamine, belongs to a type of classic primary benzylamines and is a precursor for the synthesis of bioactive compounds and the production of polyepoxides. The latest research shows that the vanillylamine has great advantages in the field of thrombolytic drug research. In plants, vanillylamine and 8-methyl-6-nonenoic acid are esterified under the catalysis of capsaicin synthase to generate capsaicin. As a synthetic active alkaloid in capsicum, capsaicin is widely used in food, drugs and other fields due to its significant anti-inflammatory and antibacterial activities. Due to special irritating characteristics, capsaicin is also used in the production of military products such as tear gas bombs. The simultaneous use of capsaicin and morphine drugs can reduce the addiction to morphine drugs. At present, the acquisition of capsaicin mainly depends on extracting from capsicum. However, due to a low content in plants and a large number of structural analogues for capsaicin, the subsequent separation and purification of capsaicin has become a great challenge. At present, vanillylamine can be produced by amination with precious metals as a catalyst and petrochemical products as a reaction substrate. However, this process is inconsistent with the requirements advocated by the green chemical industry. Studies have shown that genetically engineering Escherichia coli can produce vanillylamine using vanillic acid and ferulic acid in a lignin hydrolyzate as substrates, with a substrate conversion rate of up to 95%. However, this method is mainly limited by the concentration of products such as vanillic acid and ferulic acid in the lignin hydrolyzate, and yields up to 2.5 g/L. Moreover, a method for biosynthesizing capsaicin has not been previously reported.

### SUMMARY

In view of this, an objective of the present disclosure is to provide engineered Saccharomyces cerevisiae strains for high production of vanillylamine and capsaicin.

To achieve the above objective, the present disclosure provides the following technical solutions:

The present disclosure provides an engineered Saccharomyces cerevisiae strain for production of vanillylamine, where two gene modules are inserted into the Saccharomyces cerevisiae genome: a gene module 1 includes PAL2, 4H, 4CL2, and HCT genes, and a gene module 2 includes (CoAoMT1, FerB2, and pAMT genes.

In the present disclosure, in order to realize the efficient assembly of a biosynthetic pathway of producing vanillylamine in Saccharomyces cerevisiae (S. cerevisiae), the pathway is divided into two modules. Module 1: starting with phenylalanine, through four-step enzyme catalysis using phenylalanine lyase (PAL), cinnamic acid-4-hydroxy lase (C4H), 4-coumaroyl-CoA ligase (4CL), and hydroxyl cinnamoyltransferase (HCT), caffeoyl-CoA is obtained. Module 2: using the caffeoyl-CoA as a substrate, the vanillylamine is obtained by catalysis with caffeic acid transmethoxylase (CAMT), feruloyl-CoA lyase (FerB), and putative aminotransferase (pAMT). The modules I and II are constructed on the genome of Saccharomyces cerevisiae to realize the green fermentation and production of vanillylamine. The pattern diagram is shown in FIG. 1. Thus, the assembly of heterologous long pathways is easily and efficiently achieved in a modular method.

The present disclosure further provides a method of producing the engineered Saccharomyces cerevisiae strain, including the following steps: constructing two double transformation units of TP11t-PAL2-TDH3p-ADH1p-C4H-PG11t and ADH1t-4CL2-PGK1p-TEF2p-HCT-CYC1t: inserting the two double transformation units into a YPRCA15 site of a genome of Saccharomyces cerevisiae to obtain a strain Z1: constructing a gene expression cassette FBA1p-pAMT-ATP15t and a third double transformation unit HOG1t-CCoAoMT1-ENO2p-PYK1p-FerB2-LRP1t; and inserting the gene expression cassette and the third double transformation unit jointly into a YORWΔ17 site of a genome of the strain Z1.

The present disclosure further provides an engineered Saccharomyces cerevisiae strain for production of vanillylamine, where the engineered Saccharomyces cerevisiae is obtained by inserting pheA, Aro9, and TAL genes into the genome of the engineered Saccharomyces cerevisiae strain above. By overexpressing the shikimic acid pathway, the supply of precursors for the synthesis of vanillylamine is optimized, such that the production of vanillylamine in the engineered Saccharomyces cerevisiae is further improved. A method of producing vanillylamine includes the following steps: constructing two gene expression cassettes of PFY1p-pheA-PRC1t-ACT1p-Aro9-BAN4t and HXT7p-TAL-PRS28At; and inserting the two gene expression cassettes into a delta1 site of a genome of the engineered Saccharomyces cerevisiae strain.

The present disclosure further provides an engineered Saccharomyces cerevisiae for production of vanillylamine, where the engineered Saccharomyces cerevisiae strain is obtained by inserting SahH and SAM2 genes into the genome of any one of the engineered Saccharomyces cerevisiae strains above: alternatively, an engineered Saccharomyces cerevisiae strain is produced by inserting mtn, luxS, and SAM2 genes into the genome of any one of the engineered Saccharomyces cerevisiae strains above. In the present disclosure, the efficiency of methylation reactions involved in the synthetic pathway is improved by optimizing a regeneration ability of S-adenosyl-L-methionine (SAM). In this way, a fermentation yield of vanillylamine up to 16.48 g/L, which is the highest level of de novo biosynthesis of vanillylamine by Saccharomyces cerevisiae.

The present disclosure further provides a method for producing engineered Saccharomyces cerevisiae strains, including the following steps: constructing two gene expression cassettes of HIS-CYC1P-SahH-ALY2t and ENO2p-SAM2-SCW4t, and inserting the two gene expression cassettes into an rDNA site of a genome of any one of the engineered Saccharomyces cerevisiae strains above: alternatively; constructing two gene expression cassettes of HIS-FBA1p-mtn-NAT5t-ADH1p-luxS-IDP1t and ENO2p-SAM2-SCW4t, and inserting the two gene expression cassettes into an rDNA site of a genome of any one of the engineered Saccharomyces cerevisiae strains above.

The present disclosure further provides an engineered Saccharomyces cerevisiae strain for production of capsaicin, where the engineered Saccharomyces cerevisiae strain is produced by inserting Kas, Acl, Fat, and AT3 genes into any one of the engineered Saccharomyces cerevisiae strains above. In the present disclosure, a fatty acid branched pathway and capsaicin synthase related to capsaicin synthesis are further expressed in the engineered strains producing vanillylamine, thereby realizing the de novo biosynthesis of capsaicin. The pattern diagram is shown in FIG. 1. As shown in FIG. 1, the synthetic pathway of vanillylamine and its derivative capsaicin are divided into three modules. The three modules are constructed on the genome of Saccharomyces cerevisiae, so as to realize the de novo biosynthesis of vanillylamine and capsaicin. The present disclosure is the first time to produce capsaicin de novo by engineered Saccharomyces cerevisiae strains. The present disclosure further provides a method of producing the engineered Saccharomyces cerevisiae strains, including the following steps: constructing two double transformation units of ASP3t-Kas-PGK1p-FBA1p-Acl-HAP4t and YCP4t-Fat-GPM1p-ACT1p-AT3-MDM35t, and constructing the two double transformation units into a YORWΔ22 site of a genome of any one of the engineered Saccharomyces cerevisiae strains.

The present disclosure further provides use of any one of the engineered Saccharomyces cerevisiae strains in production of vanillylamine.

The present disclosure further provides use of any one of the engineered Saccharomyces cerevisiae strains in production of capsaicin.

The present disclosure has the following beneficial effects:

In the present disclosure, a synthesis pathway of vanillylamine is expressed heterologously in engineered Saccharomyces cerevisiae strains for the first time, and biosynthesis of the vanillylamine is realized for the first time. After the optimization of precursors supply and the optimization of S-adenosyl-L-methionine (SAM) cycle, a yield of the vanillylamine reaches 16.48 g/L, and a synthesis efficiency of the vanillylamine is increased by nearly 150%. This is the highest optimization efficiency obtained by optimizing a SAM cofactor strategy so far.

Based on the synthesis of vanillylamine by the engineered Saccharomyces cerevisiae strains, a synthesis pathway of 8-methyl-6-nonenoyl-coenzyme A is constructed heterologously, and a capsaicin synthase AT3 is expressed heterologously. In this way, de novo total biosynthesis of the capsaicin is realized for the first time, and a yield of the capsaicin is 80.23 μg/L.

In the present disclosure, the engineered Saccharomyces cerevisiae strains directly generate vanillylamine and capsaicin by glucose metabolism, which has enabled the de novo biosynthesis of the bulk chemical vanillylamine, and its complex derivative capsaicin. These engineered strains use a constitutive promoter and a strong terminator to control the high-level expression of heterologous genes, without adding inducers or additional high-priced substrates. In this way, a fermentation process is simplified, a production cost is reduced, and the vanillylamine and capsaicin can be biosynthesized by direct fermentation, compared with chemical synthesis, this technology is more in line with the concept of green and sustainable development.

### BRIEF DESCRIPTION OF THE DRAWINGS

FIG. 1 shows a hierarchical design schema diagram of the approaches for vanillylamine and capsaicin;

FIG. 2 shows output results of the vanillylamine of engineered Saccharomyces cerevisiae strains constructed in Examples 1 to 4;

FIG. 3A and FIG. 3B show HPLC-MS/MS quantitative analysis results of capsaicin produced by the fermentation of the C1 strain; FIG. 3A shows the HPLC-MS/MS diagram of a capsaicin standard sample, and FIG. 3B shows the HPLC-MS/MS diagram of capsaicin synthesized by an engineered yeast strain.

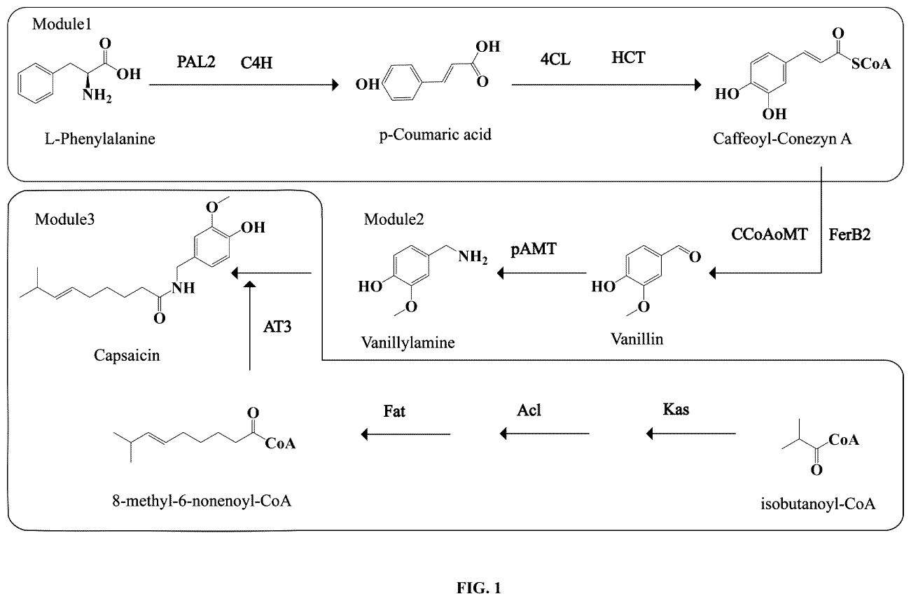

### DETAILED DESCRIPTION OF THE EMBODIMENTS

The technical solution provided by the present disclosure will be described in detail below with reference to examples, which should not be construed as limiting the protection scope of the present disclosure.

In the following examples, all methods are conventional methods unless otherwise specified.

All materials and reagents used in the following examples may be commercially available unless otherwise specified.

### Example 1

Construction of engineered Saccharomyces cerevisiae strain for vanillylamine synthesis
1) PAL2, C4H, and 4CL2 genes from Arabidopsis, HCT and CCoAoMT1 genes from tobacco, a FerB2 gene from Sphingomonas, and a pAMT gene from pepper were selected. The coding genes of the above-mentioned genes were codon-optimized and chemically synthesized by GENEWIZ Company. The synthesized PAL2 had a nucleotide sequence shown in SEQ ID NO: 1, the C4H had a nucleotide sequence shown in SEQ ID NO: 2, the 4CL2 had a nucleotide sequence shown in SEQ ID NO: 3, the HCT had a nucleotide sequence shown in SEQ ID NO: 4, the CCoAoMT1 had a nucleotide sequence shown in SEQ ID NO: 5, the FerB2 had a nucleotide sequence shown in SEQ ID NO: 6, and the pAMT had a nucleotide sequence shown in Shown in SEQ ID NO: 7.
2) The constitutive promoters TDH3p, ADH1p, PGK1p, and TEF2p in Saccharomyces cerevisiae were selected, and two head-to-head promoter modules were constructed with a genome of Saccharomyces cerevisiae as a template by OE-PCR using the primer sequences 1 to 8. The two promoter modules were saved into a plasmid. A specific process was as follows:
a: A reaction system included: 60 ng of purified and recovered fragments of TDH3p, 60 ng of purified and recovered fragments of ADH1p, 2 μL of dNTP (10 mM), 1 μL of PrimeSTAR DNA polymerase, 10 μL of 5×PrimeSTAR Buffer, and add water to make up to 50 μL; PCR was conducted by a PCR instrument: 98° C. for 3 min; 98° C. for 10 s, 65° C. for 1 min (1 Kb/min), cycled 30 times; 68° C. for 5 min, and stored at 4° C. b: After the reaction, Tiangen's Pfu DNA polymerase was used, the above-mentioned reaction solution without purification was used as a template, and conventional PCR amplification was conducted with primers 1 and 4 to obtain a TDH3p+ADH1p-connected fragment. c: After purification and recovery, 50 ng of the TDH3p+ADH1p fragment was selected (the amount was determined by a length of the gene fragment, 50 ng/Kb), 1 μL of pBM16A Vevtor and 1 μL of 10×Toposmart were added (the reagent was a pBM16A Toposmart cloning kit was purchased from Biomed Company) box), and water was added to make up to 10 μL; a resulting system was mixed evenly, and PCR was conducted at 25° C. for 30 min. d: After the reaction, 10 μL of an obtained reaction solution was added to 100 μL of Escherichia coli competent cells melted on ice, mixed gently, and kept in ice bath for 30 min. e: After the cells were heat-shocked at 42° C. for 90 s, they were immediately transferred to ice water for 2 min. f: 900 μL of LB medium was added to the competent cells, and cultured with shaking at 200 rpm for 1 h at 37° C. g: Centrifugation was conducted at 4000 rpm for 1 min, 900 μL of supernatant was removed, the cells were resuspended with the remaining liquid, spread evenly on an LB plate containing 100 pg/L Amp, cultured overnight at 37° C., colony PCR was conducted to verify correct single colony, and a plasmid was extracted from the correct single colony and then sent to GENEWIZ for sequencing. In this way, the TDH3p+ADH1p fragment was stored in a plasmid pBM16A for later use (pBM16A-TDH3p+ADH1p); steps a to g were repeated to save a prepared spare plasmid pBM16A-PGK1p+TEF2p.
3) TPI1t, PGI1t, ADH1t, and CYC1t were assembled into two back-to-back terminator modules (a Sap1 restriction site was inserted between the two terminators and homology arms L1-L2, L3-L4 were added at both ends). The functional genes were amplified by PCR using primers 9 to 16 (the functional genes were all based on the plasmid synthesized by entrusting GENEWIZ as a template), and the Sap1 restriction sites corresponding to the promoter module and the terminator module were assembled at both ends.

The primer sequences involved in the above process included:
Primer 1: 5′>CCTGCTCTTCACATTTTGTTTATGTGTGTTTATTC<3′ (SEQ ID NO: 51), the upstream primer for the amplification of promoter TDH3p, the underlined sequence was a cohesive end formed after the promoter TDH3p was digested by Sap1.
Primer 2: 5′>TCTGTTCTATTGTATATCTCCCCTCCGCCACCTACATGTATAC TAGCGTTGAATGTTAG<3′ (SEQ ID NO: 52), the promoter TDH3p for the amplification of downstream primer, the underlined sequence was an overlapping sequence with the promoter ADH1p.
Primer 3: 5′>AAACTTCTTGTTGTTGACGCTAACATTCAACGCTAGTATAC ATGTAGGTGGCGGAGGGG<3′ (SEQ ID NO: 53), the upstream primer for the amplification of promoter ADH1p, the underlined sequence was an overlapping sequence with the promoter TDH3p.
Primer 4: 5′>CCTGCTCTTCATGTATATGAGATAGTTGATTGTATG<3′ (SEQ ID NO: 54), the downstream primer for the amplification of promoter ADH1p, the underlined sequence was a cohesive end formed after the promoter ADH1p was digested by Sap1.
Primer 5: 5′>CCTGCTCTTCACATTGTTTTATATTTGTTGTAAAAAG<3′ (SEQ ID NO: 55), the upstream primer for the amplification of promoter PGK1p, the underlined sequence was a cohesive end formed after the promoter PGK1p was digested by Sap1.
Primer 6: 5′>GGAAGCGCCTACGCTTGACATCTACTATATGTAAGTATAAC GCACAGATATTATAACAT<3′ (SEQ ID NO: 56), the downstream primer for the amplification of promoter PGK1p, the underlined sequence was an overlapping sequence with the promoter TEF2p.
Primer 7: 5′>GCAAATGCCTATTGTGCAGATGTTATAATATCTGTGCGTTA TACTTACATATAGTAGAT<3′ (SEQ ID NO: 57), the upstream primer for the amplification of promoter TEF2p, the underlined sequence was an overlapping sequence with the promoter PGK1p.
Primer 8: 5′>CCTGCTCTTCATGTGGTACTAGTGTTTAGTTAATTATAG<3′ (SEQ ID NO: 58), the downstream primer for the amplification of promoter TEF2p, the underlined sequence was a cohesive end of promoter TEF2p after Sap1 digestion.
Primer 9: 5′>CCTGCTCTTCAGGTTTAACAAATTGGAATAGGAGCACCGT TC<3′ (SEQ ID NO: 59), the upstream primer for gene PAL2 amplification, the underlined sequence was a cohesive end of gene PAL2 after Sap1 digestion.
Primer 10: 5′>CCTGCTCTTCAGTAATGGACCAAATTGAGGCAATGTTG<3′ (SEQ ID NO: 60), the downstream primer for gene PAL2 amplification, the underlined sequence was a cohesive end of gene PAL2 after Sap1 digestion.
Primer 11: 5′>CCTGCTCTTCAACA ATGGACTTGTTGTTGTTGGAAAAG<3′ (SEQ ID NO: 61), the upstream primer for gene C4H amplification, the underlined sequence was a cohesive end of gene C4H after Sap1 digestion.
Primer 12: 5′>CCTGCTCTTCAGATTTAACAATTTCTTGGCTTCATAAC<3′ (SEQ ID NO: 62), downstream primers for gene C4H amplification, the underlined sequence was a cohesive end of gene C4H after Sap1 digestion.
Primer 13: 5′>CCTGCTCTTCAGGT TTAACATAACTTAGCTTTTAAATC<3′ (SEQ ID NO: 63), the upstream primer for gene 4CL2 amplification, the underlined sequence was a cohesive end of gene 4CL2 after Sap1 digestion.
Primer 14: 5′>CCTGCTCTTCAGTAATGATTACTGCAGCATTGCACG<3′ (SEQ ID NO: 64), downstream primer for gene 4CL2 amplification, the underlined sequence was a cohesive end of gene 4CL2 after Sap1 digestion.
Primer 15: 5′>CCTGCTCTTCAACAATGAAAATTGAAGTTAAAGAATC<3′ (SEQ ID NO: 65), the upstream primer for gene HCT amplification, the underlined sequence was a cohesive end of gene HCT after Sap1 digestion.
Primer 16: 5′>CCTGCTCTTCAGAT TTAAAAATCGTACAAAAATTTCTCA AAC<3′ (SEQ ID NO: 66), the downstream primer for gene HCT amplification, the underlined sequence is the cohesive end of gene HCT after Sap1 digestion.
4) Using a Golden Gate ligation method, and using a terminator module as a receiving plasmid (0.2 μmol), the promoter module and two functional genes (each 0.6 μmol) were inserted. The Golden Gate included:

The reaction was completed in a 10 μL system, including: 2.5 μL Anza T4 DNA Ligase Master Mix, 0.5 μL Anza10× Buffer, 0.2 μmol acceptor vector, inserted fragment 0.6 μmol, 0.5 μL Anza 36 Eco31I, and adding sterile water to make up to 10 μL. All enzymes were purchased from Thermo Fisher. The Golden Gate reaction was conducted in a PCR instrument: 37° C. for 5 min; 37° C. for 1 min, 23° C. for 1 min, 30 cycles of reaction; 37° C. for 5 min; 65° C. for 10 min; stored at 4° C. 10 μL of the assembly mixture was transformed into E. coli DH5u competent cells and incubated overnight at 37° C. on an LB agar plate with Amp antibiotics. 50 colonies were randomly selected from the LB agar plate with 2×Taq Master Mix (TIANGEN) for PCR verification, and correct colonies were selected. Two double transformation units DH5u/PUC57T-(TPI1t-PAL2-TDH3p-ADH1p-C4H-PGI1t) and DH5u/PUC57T-(ADH1t-4CL2-PGK1p-TEF2p-HCT-CYC1t) were assembled by the above method.
5) According to the following steps, the two double transformation units (obtained in step 4) containing PAL2, C4H, HCT, and 4CL2 were inserted into a YPRCA15 site of the genome of Saccharomyces cerevisiae to realize the assembly of module 1 to form a strain Z1:
   a. Preparation of Competent Cells of Saccharomyces cerevisiae
(1) A single clone was selected on a solid plate, transferred to a YPD liquid medium without antibiotics, and cultured overnight in a shaker at 30° C. and 200 rpm. (2) The cultured bacterial solution was transferred to a fresh YPD liquid medium without antibiotics at a volume of 10%, and continued to culture in a shaker at 30° C. and 200 rpm for 5-8 h. (3) 1.5 ml of the bacterial solution was placed in a centrifuge tube, centrifuged at 5000 rpm for 3 min, and a supernatant was discarded. (4) The cells were resuspended in 1 ml of sterile water, centrifuged at 5000 rpm for 2 min, and a supernatant was discarded. (5) The cells were resuspended with 1 mL of 100 mM LiAc, allowed to stand for 3 min, centrifuged at 4000 rpm for 4 min, 900 μl of the supernatant was discarded, the cells were resuspended, and placed on ice. The Saccharomyces cerevisiae competent cells were prepared.
   b. Transformation of Saccharomyces Cerevisiae

(1) 40 μL of 10% salmon sperm single-stranded ssDNA was denatured in boiling water for 5 min, and immediately inserted into ice for later use. (2) 500 ng for each of the two double transformation units prepared in 4) were added to a new centrifuge tube, 500 ng of the plasmid (Δ15 site1) containing the upper end sequence of the YPRC Δ15 site and the selection marker gene, and the plasmid (Δ15 site2) containing the upper end sequence of the YPRC Δ15 site were added with 40 uL of denatured 10% salmon sperm single-stranded ssDNA. A mixture of the above two was transferred to S. cerevisiae competent cells placed on ice, and 50% w/v PEG 3350 480 μL and 1 M LiAc 72 μL were added sequentially. The centrifuge tube was vortexed at high speed for 1 min to fully mix the components. (3) A transformation system was put in a 30° C. incubator and allowed to stand for 30 min. (4) 72 μL dimethyl sulfoxide (DMSO) was added, and vortexed to mix well. (5) The centrifuge tube was placed in a 42° C. water bath, heat-shocked for 10 min, centrifuged at 4000 rpm for 5 min, and a supernatant was discarded. (6) 200 μL of 5 mM CaCl₂) was added, the cells were resuspended and mixed well, allowed to stand for 10 min, centrifuged at 4000 rpm for 5 min, and a supernatant was discarded. (7) 1 mL of sterile water was added to resuspend and wash the cells, centrifuged at 4000 rpm for 1 min, a supernatant was discarded; then 100 uL of sterile water was added to resuspend the cells, and applied to the corresponding solid medium. (8) inverted culture was conducted in a constant-temperature incubator at 30° C. for 2-4 d.
6) On the basis of the strain Z1, the three coding genes CCoAoMT1, FerB2, and pAMT were constructed into aYORWΔ17 site of the genome of Saccharomyces cerevisiae by the method of 5). In this way, the assembly of module 2 in Saccharomyces cerevisiae was realized, and a final engineered Saccharomyces cerevisiae strain (Z2) was generated. In this step, ENO2p, PYK1p, FBA1p promoters and HOG1t, LRP1t, ATP15t terminators were selected to control gene expression. Since there were only three genes in this module, pAMT directly followed the OE-PCR method in step 2) to assemble the pAMT gene expression cassette: FBA1p-pAMT-ATP15t. A double transformation unit of CCoAoMT1 and FerB2 was constructed by the method in step 4): HOG1t-CCoAoMT1-ENO2p-PYK1p-FerB2-LRP1t.

The gene sequence of strain Z2 constructed by the above method was based on the YORWΔ15 site in the genome of Saccharomyces cerevisiae: URA3-TPIt-PAL2-TDH3p-ADH1p-C4H-PGI1t-ADH1t-4CL2-PGKp-TEF2p-HCT-CYC1t;

YORWΔ17 site was: TEF1p-hphNT1-CYC1t-HOG1t-CCoAoMT1-ENO2p-PYK1p-FerB2-LRP1t-FBA1p-pAMT-ATP15t.

In step 6), the primer sequences involved were as follows:
Primer 17: 5′>CCTGCTCTTCACATGGTCTCACATCTATTATTGTATG<3′ (SEQ ID NO: 67), the upstream primer for the amplification of promoter ENO2p, the underlined sequence was a cohesive end formed after the promoter ENO2p was digested by Sap1.
Primer 18: 5′>TTGTACATACTAATATATATAAACAAAGTAACGTCTCCT ACGCGGCGTTATGTCACTAA<3′ (SEQ ID NO: 68), the downstream primer for amplification of promoter ENO2p, the underlined sequence was an overlapping sequence with the promoter PYK1p.
Primer 19: 5′>CGCAAGTTGGTGCACGTCGTTAGTGACATAACGCCGCGT AGGAGACGTTACTTTGTTTA<3′ (SEQ ID NO: 69), the upstream primer for the amplification of the promoter PYK1p, the underlined sequence is the overlapping sequence with the promoter ENO2p.
Primer 20: 5′>CCTGCTCTTCATGTATAATTTGCGATCGTAGTTGGG<3′ (SEQ ID NO: 70), the downstream primer for the amplification of promoter PYK1p, the underlined sequence was a cohesive end formed after the promoter PYK1p was digested by Sap1.
Primer 21: 5′>CCTGCTCTTCAGGT TTAAGATATTCTTCTGCACAAAG<3′ (SEQ ID NO: 71), the upstream primer for gene CCoAoMT1 amplification, the underlined sequence was a cohesive end of gene CCoAoMT1 after Sap1 digestion.
Primer 22: 5′>CCTGCTCTTCAGTAATGGCTACTAACGGTAGACACCAAG<3′ (SEQ ID NO: 72), the downstream primer for gene CCoAoMT1 amplification, the underlined sequence was a cohesive end of gene CCoAoMT1 after Sap1 digestion.
Primer 23: 5′>CCTGCTCTTCAACA ATGTCTGATGAATTGACATTTG<3′ (SEQ ID NO: 73), the upstream primer for gene FerB2 amplification, the underlined sequence was a cohesive end of gene FerB2 after Sap1 digestion.
Primer 24: 5′>CCTGCTCTTCAGATTTAATCTTCTCTTCTATAACCAC<3′ (SEQ ID NO: 74), the downstream primer for gene FerB2 amplification, the underlined sequence was a cohesive end of gene FerB2 after Sap1 digestion.
Primer 25: 5′>CTTGTATTGTTCTAACGATCCGACCATTTCATCCTGAAT ATAACAATACTGACAGTACT<3′ (SEQ ID NO: 75), the upstream primer for the amplification of the promoter FBA1p, the underlined sequence was an overlapping sequence with the terminator LRP1t.
Primer 26: 5′>CATATCATGACCCATAAATTCGTTTGTAATATTTGCCAT TTTGAATATGTATTACTTGG<3′ (SEQ ID NO: 76), the downstream primer for the amplification of the promoter FBA1p, the underlined sequence was an overlapping sequence with the gene pAMT.
Primer 27: 5′>TTGTCATATATAACCATAACCAAGTAATACATATTCAAA ATGGCAAATATTACAAACGA<3′ (SEQ ID NO: 77), the upstream primer for gene pAMT amplification, the underlined sequence was an overlapping sequence with the promoter FBA1p.
Primer 28: 5′>CGAGTAAAAAAAGAGCTGCAGTTCCCAGGAAGCGTTAAA TTATTGTTTTTGTGACTTCA<3′ (SEQ ID NO: 78), the downstream primer for gene pAMT amplification, the underlined sequence is the overlapping sequence with the terminator AT915t.
Primer 29: 5′>GAAAAAAGAGTTGAAGAATTGAAGTCACAAAAACAATAA TTTAACGCTTCCTGGGAACT<3′ (SEQ ID NO: 79), the upstream primer for the amplification of terminator ATP15t, the underlined sequence was an overlapping sequence with the gene pAMT.
Primer 30: 5′>GTGATGAATTTTGAGAGCCCACTTTTGTTGGGGACGATT GAGAGGCTGAAGGCAGAGAA<3′ (SEQ ID NO: 80), the downstream primer for the amplification of terminator ATP15t, the underlined sequence was an overlapping sequence with the YORWΔ17 site.

The nucleotide sequences of the promoter and terminator involved in the above process were: TDH3p (SEQ ID NO: 8), ADH1p (SEQ ID NO: 9), PGK1p (SEQ ID NO: 10), TEF2p (SEQ ID NO: 11), TPI1t (SEQ ID NO: 12), PGI1t (SEQ ID NO: 13), ADH1t (SEQ ID NO: 14), CYC1t (SEQ ID NO: 15), ENO2p (SEQ ID NO: 16), PYK1p (SEQ ID NO: 17), FBA1p (SEQ ID NO: 18), HOG1t (SEQ ID NO: 19), LRP1t (SEQ ID NO: 20), ATP15t (SEQ ID NO: 21).
7) Z2 strain fermentation test: a single colony verified by PCR was selected from the plate, and inoculated into 20 mL of YPD medium (2% glucose, 2% peptone, 1% yeast powder), cultured at 220 rpm and 30° C. for 36 h to obtain a seed solution. The seed solution was inoculated into a 1 L fermenter with 600 mL of medium at an inoculation amount of the initial OD₆₀₀ value of 0.1, and then cultured at a pH value of 7.0 and 30° C. under a ventilation flow at 0.1 and a rotational speed at 170 rpm for 96 h. 24 h after inoculation, 6 g of glucose was supplemented every 12 h until the end of fermentation, and samples were taken every 24 h. 1 mL of 1 M HCl was added into 5 mL of a fermentation broth, heated to boil for 10 min to fully lyse the bacterial cells. Centrifugation was conducted to remove bacterial cells, 1 mL of supernatant was added with an equal volume of methanol to remove salt, and then subjected to HPLC for qualitative and quantitative analysis to determine the content of vanillylamine. The results showed that the content of vanillylamine reached 6.47 g/L after the fermentation of engineered Saccharomyces cerevisiae strain.

### Example 2

Modification of yeast intrinsic pathway to enhance vanillylamine production
1) The coding gene pheA (nucleotide sequence shown in SEQ ID NO: 22) of bifunctional chorismate mutase prebenzoate dehydratase derived from E. coli and the transaminase coding gene Aro9 of Saccharomyces cerevisiae (nucleotide sequence shown in SEQ ID NO: 23), and the PFY1p promoter (nucleotide sequence shown in SEQ ID NO: 24), ACT1p promoter (nucleotide sequence shown in SEQ ID NO: 25), as well as the PRC1t terminator (nucleotide sequence shown in SEQ ID NO: 26), and BAN4t terminator (nucleotide sequence shown in SEQ ID NO: 27) terminator were assembled into a gene expression cassette using the method of OE-PCR in Example 1: PFY1p-pheA-PRC1t-ACT1p-Aro9-BAN4t.

The primer sequences used in the above steps were as follows (both promoter and terminator used the genome of Saccharomyces cerevisiae as a template; the target gene pheA used the genome of E. coli MG1655 as a template; the target gene Aro9 used the genome of Saccharomyces cerevisiae as a template):
Primer 31: 5′>GCATCCACATCTTCACATGAAAATAGAGGCCAATCAGGC AGGAGACGTTACTTTGTTTA<3′ (SEQ ID NO: 81), the upstream primer for the amplification of promoter PFY1p, the underlined sequence was an overlapping sequence with the terminator PRS28At.
Primer 32: 5′>TTTCTCTCGCAGCGCCAGTAACGGGTTTTCCGATGTCAT ATAATTTGCGATCGTAGTTG<3′ (SEQ ID NO: 82), the downstream primer for the amplification of the promoter PFY1p, the underlined sequence was an overlapping sequence with the gene pheA.
Primer 33: 5′>TACATACACAACATAAACCCAACTACGATCGCAAATTAT ATGACATCGGAAAACCCGTT<3′ (SEQ ID NO: 83), the upstream primer for gene pheA amplification, the underlined sequence was an overlapping sequence with the promoter FBA1p.
Primer 34: 5′>CTGATAATAAAAACGGTATGCCTACACATACACGCTTTA TCAGGTTGGATCAACAGGCA<3′ (SEQ ID NO: 84), the downstream primer for gene pheA amplification, the underlined sequence was an overlapping sequence with the terminator PRC1t.
Primer 35: 5′>TACCCAAGTGAGAACGTAGTGCCTGTTGATCCAACCTGA TAAAGCGTGTATGTGTAGGC<3′ (SEQ ID NO: 85), the upstream primer for amplification of the terminator PRC1t, the underlined sequence was an overlapping sequence with the gene pheA.
Primer 36: 5′>ATTATATTATATGGGTCTGCAAGGTAGAGGCGCGCTTGT GCAGCGATCAGCAATAATGA<3′ (SEQ ID NO: 86), downstream primer for terminator PRC1t amplification, the underlined sequence was an overlapping sequence with the promoter ACT1p.
Primer 37: 5′>ATTCCGCGTTTGCTTCATTTCATTATTGCTGATCGCTGC ACAAGCGCGCCTCTACCTTG<3′ (SEQ ID NO: 87), the upstream primer for the amplification of promoter ACT1p, the underlined sequence was an overlapping sequence with the terminator PRC1t.
Primer 38: 5′>GGAAGTGTAATCAACAGGGGGGGCAGAACCAGCAGTCAT TGTTAATTCAGTAAATTTTC<3′ (SEQ ID NO: 88), the downstream primer for the amplification of promoter ACT1p, the underlined sequence was an overlapping sequence with gene aro9.
Primer 39: 5′>CTTTTTTCTTCCCAAGATCGAAAATTTACTGAATTAACA ATGACTGCTGGTTCTGCCCC<3′ (SEQ ID NO: 89), the upstream primer for gene aro9 amplification, the underlined sequence was an overlapping sequence with the promoter ACT1p.
Primer 40: 5′>CCTCTCCTAGCGTACACGGATGGCAAGAATAAACTGGCT TCAACTTTTATAGTTGTCAA<3′ (SEQ ID NO: 90), the downstream primer for gene aro9 amplification, the underlined sequence was an overlapping sequence with the terminator BNA4t.
Primer 41: 5′>AGTGGCATAAAAGAATTTTTTGACAACTATAAAAGTTGA AGCCAGTTTATTCTTGCCAT<3′ (SEQ ID NO: 91), the upstream primer for amplification of the terminator BNA4t, the underlined sequence was an overlapping sequence with the gene pheA.
Primer 42: 5′>AGCTGAAATGCAAGGATTGATAAAATAATAGGATAATA GAAGAATGGATCAAAATGGC<3′ (SEQ ID NO: 92), the downstream primer for amplification of the terminator PRC1t, the underlined sequence was an overlapping sequence with the delta1 site.
2) The coding gene TAL (nucleotide sequence shown in SEQ ID NO: 28) of tyrosine lyase derived from Arabidopsis thaliana, and the promoter HXT7p (nucleotide sequence shown in SEQ ID NO: 29) and the terminator PRS28At (nucleotide sequence shown in SEQ ID NO: 30) were assembled into a gene expression cassette HXT7p-TAL-PRS28At using the OE-PCR method in Example 1. The obtained gene expression cassette and the gene expression cassette assembled in step 1) were jointly constructed into the delta1 site of the genome of the strain Z2 obtained in Example 1 through lithium acetate transformation (same as step 5) in Example 1), to strengthen the precursor supply for synthesis of vanillylamine. The obtained engineered Saccharomyces cerevisiae strain was named Z3. The gene sequence of strain Z3 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-FBA1p-pAMT-ATP15t
Delta1:LEU-HXT7p-TAL-PRS28At-PFY1p-pheA-PRC1t-HXT7p-Aro9-BAN4t.

The primer sequences involved in the above steps were as follows (both the promoter and the terminator used the genome of Saccharomyces cerevisiae as a template; the target gene TAL used a gene plasmid synthesized by entrusting GENEWIZ as a template; an LEU selection marker used an LEU selection marker plasmid purchased from BIOFENG as a template):
Primer 43: 5′>ATAACATCTTTTTTTTATTCCTTCTTTGATATTTTGTCA aactgtgggaatactcaggt<3′ (SEQ ID NO: 93), the upstream primer for LEU screening marker amplification, the underlined sequence was an overlapping sequence with the delta1 site.
Primer 44: 5′>AACACGCAGGGGCCCGAAATTGTTCCTACGAGAAGTAGT TGCTTACCTGTATTCCTTTA<3′ (SEQ ID NO: 94), the downstream primer for LEU screening marker amplification, the underlined sequence was an overlapping sequence with the promoter HXT7p.
Primer 45: 5′>AGGAGAAAAAGGAGGATAGTAAAGGAATACAGGTAAGCA ACTACTTCTCGTAGGAACAA<3′ (SEQ ID NO: 95), the upstream primer for the amplification of promoter HXT7p, the underlined sequence was an overlapping sequence with the LEU screening marker.
Primer 46: 5′>AGACAATCTGTCAGCTTGTCTTTCAACAACTTGAGTCAT TTTTTAATTTTAATCAAAAA<3′ (SEQ ID NO: 96), the downstream primer for the amplification of the promoter HXT7p, the underlined sequence was an overlapping sequence with the gene TAL.
Primer 47: 5′>AACACAAAAACAAAAAGTTTTTTTAATTTTAATCAAAAA ATGACTCAAGTTGTTGAAAG<3′ (SEQ ID NO: 97), the upstream primer for gene TAL amplification, the underlined sequence was an overlapping sequence with the promoter FBA1p.
Primer 48: 5′>TTTTGTATAGCTGCAACCTTCAATCTGCAAATAAGCTTC TTAACCGAAGTCCTTACCGT<3′ (SEQ ID NO: 98), the downstream primer for gene TAL amplification, the underlined sequence was an overlapping sequence with the terminator PRS28At.
Primer 49: 5′>TTTGTTGGTTGACGAAGCTGACGGTAAGGACTTCGGTTAA GAAGCTTATTTGCAGATTGA<3′ (SEQ ID NO: 99), the upstream primer for the amplification of the terminator PRS28At, the underlined sequence was an overlapping sequence with the gene TAL.
Primer 50: 5′>TTGTACATACTAATATATATAAACAAAGTAACGTCTCCT GCCTGATTGGCCTCTATTTT<3′ (SEQ ID NO: 100), the downstream primer for amplification of the terminator PRS28At, the underlined sequence is the overlapping sequence with the promoter PFY1p.
3) The strain Z3 was fermented by the method of step 7) in Example 1, and the yield of vanillylamine increased to 7.16 g/L, which was 11% higher than that of the unoptimized strain.

### Example 3

Reconstruction of the SAM cycle to optimize the synthesis of vanillylamine
1) In the present disclosure, in order to optimize the synthesis of vanillylamine, primers 51 and 52 were used to amplify the coding gene sahH of SAH hydrolase using the genome of Saccharomyces cerevisiae as a template (nucleotide sequence shown in SEQ ID NO: 31). Primer 53 and primer 54 were used to amplify the gene SAM2 (nucleotide sequence shown in SEQ ID NO: 32) encoding SAM synthase derived from Arabidopsis thaliana, and the gene was chemically synthesized by GENEWIZ after codon optimization.

The primer sequences involved were as follows:
Primer 51: 5′>TATAGACACACAAACACAAATACACACACTAAATTAATA ATGTCTGCTCCAGCTCAAAA<3′ (SEQ ID NO: 101), the upstream primer for gene sahH amplification, the underlined sequence was an overlapping sequence with the promoter CYC1p.
Primer 52: 5′>TAAGAACAGGAAGGGATGACGTAGGAACCTTGTGCTAGA TCAATATCTGTAGTGGTCGG<3′ (SEQ ID NO: 102), the downstream primer for gene sahH amplification, the underlined sequence was an overlapping sequence with the terminator ALY2t.
Primer 53: 5′>CATAACACCAAGCAACTAATACTATAACATACAATAATA ATGGAAACTTTCTTGTTCAC<3′ (SEQ ID NO: 103), the upstream primer for gene SAM2 amplification, the underlined sequence was an overlapping sequence with the promoter ENO2p.
Primer 54: 5′>ATCAAGATTTTTTTTGTTTTTTTTTACAATTGAGTAGAC TTAAGCTTGTGGCTTGTCCC<3′ (SEQ ID NO: 104), the downstream primer for gene SAM2 amplification, the underlined sequence was an overlapping sequence with the terminator SCW4t. (Taking a gene plasmid synthesized by entrusting GENEWIZ as a template)
2) The amplification was conducted with the following corresponding primers using the genome of Saccharomyces cerevisiae as a template, to obtain promoters: CYC1p (nucleotide sequence shown in SEQ ID NO: 33), ENO2p (nucleotide sequence shown in SEQ ID NO: 16); terminators: ALY2t (nucleotide sequence shown in SEQ ID NO: 34), SCW4t (nucleotide sequence shown in SEQ ID NO: 35). The primers 55 and 56, PCR-amplified HIS screening marker gene, and the gene fragments amplified in the above step were assembled into gene expression cassettes according to the processes a to b in step 3) of Example 1: HIS-CYC1p-sahH-ALY2t; ENO2p-SAM2-SCW4t. The two gene expression cassettes were constructed into the rDNA sites of the genomes of strains Z2 and Z3 by the transformation method in 5) in Example 1 to form strains Z4 and Z7, respectively.

The gene sequence of strain Z4 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-FBA1p-pAMT-ATP15t
rDNA:HIS-CYC1p-sahH-ALY2t-ENO2p-SAM2-SCW4t

The gene sequence of strain Z7 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-FBA1p-pAMT-ATP15t
Delta1:LEU-HXT7p-TAL-PRS28At-PFY1p-pheA-PRC1t-HXT7p-Aro9-BAN4t.
rDNA:HIS-CYC1P-sahH-ALY2t-ENO2p-SAM2-SCW4t.

The primer sequences involved in the above were: the promoter and the terminator were both based on the Saccharomyces cerevisiae genome as a template, and the special templates were marked below:
Primer 55: 5′>ACCCTGCCCTCATATCACCTGCGTTTCCGTTAAACTATC CTAGTACACTCTATATTTTT<3′ (SEQ ID NO: 105), the upstream primer for amplifying the HIS screening marker gene, the underlined sequence was an overlapping sequence with the rDNA site.
Primer 56: 5′>GCCAAGTAGGCAATTATTTAGTACTGTCAGTATTGTTAT CCTGATGCGGTATTTTCTCC<3′ (SEQ ID NO: 106), the downstream primer for amplifying the HIS screening marker gene, the underlined sequence was an overlapping sequence with the promoter CYC1p. (Taking the HIS screening marker plasmid purchased from BIOFENG as a template)
Primer 57: 5′>ATACCGCACAGATGCGTAAGGAGAAAATACCGCATCAGG AATTTTTTTGGAAAACCAAG<3′ (SEQ ID NO: 107), the upstream primer for CYC1p promoter amplification, the underlined sequence was an overlapping sequence with the HIS screening marker gene.
Primer 58: 5′>GATATCAGCGATTTTGTAGTTTTGAGCTGGAGCAGACAT TATTAATTTAGTGTGTGTAT<3′ (SEQ ID NO: 108), the downstream primer for CYC1p promoter amplification, the underlined sequence was an overlapping sequence with the gene sahH.
Primer 59: 5′>CTCCTTTATTTGTTCTTTGTCTTTTGACTTCTTCCCGCT ACGCGGCGTTATGTCACTAA<3′ (SEQ ID NO: 109), the upstream primer for the amplification of the promoter ENO2p, the underlined sequence was an overlapping sequence with the terminator ALY2t.
Primer 60: 5′>TTCGTTAACAGATTCAGAAGTGAACAAGAAAGTTTCCAT TATTATTGTATGTTATAGTA<3′ (SEQ ID NO: 110), the downstream primer for amplification of the promoter ENO2p, the underlined sequence was an overlapping sequence with the gene SAM2.
Primer 61: 5′>GAAGAAGGTCCATTCAAGGCCGACCACTACAGATATTGA TCTAGCACAAGGTTCCTACG<3′ (SEQ ID NO: 111), the upstream primer for amplification of the terminator ALY2t, the underlined sequence was an overlapping sequence with the gene sahH.
Primer 62: 5′>CGCAAGTTGGTGCACGTCGTTAGTGACATAACGCCGCGT AGCGGGAAGAAGTCAAAAGA<3′ (SEQ ID NO: 112), the downstream primer for amplification of the terminator ALY2t, the underlined sequence was an overlapping sequence with the promoter ENO2p.
Primer 63: 5′>GTTGTTAAGCCATTGAAGTGGGACAAGCCACAAGCTTAA GTCTACTCAATTGTAAAAAA<3′ (SEQ ID NO: 113), the upstream primer for terminator SCW4t amplification, the underlined sequence was an overlapping sequence with the gene SAM2.
Primer 64: 5′>AGACTGTCAAGGAGGGTATTCTGGGCCTCCATGTCGCTG CCAAGAGGATCGAGAAACCA<3′ (SEQ ID NO: 114), downstream primer for terminator SCW4t amplification, the underlined sequence was an overlapping sequence with the rDNA site.
3) The strains Z4 and Z7 were subjected to fermentation culture using the method of step 7) in Example 1, and the yields of vanillylamine were increased to 9.21 g/L and 14.89 g/L, which showed increases of 47% and 130% on the vanillylamine compared with the unoptimized strains, respectively.

### Example 4

Optimizing the SAH hydrolysis pathway, optimizing the regeneration ability of SAM cycle, and improving the synthesis of vanillylamine
1) PCR amplification was conducted on mtn (nucleotide sequence shown in SEQ ID NO: 36) and luxS (nucleotide sequence shown in SEQ ID NO: 37) using two pairs of primers: primers 65 and 66, and primers 67 and 68, respectively, with a genome of E. coli as a template. Primers 53 and 54 were used to amplify the gene SAM2 (nucleotide sequence shown in SEQ ID NO: 32) derived from Arabidopsis thaliana for encoding SAM synthetase.

The primer sequences involved in the above steps were as follows:
Primer 65: 5′>TTGTCATATATAACCATAACCAAGTAATACATATTCAAA ATGAAAATCGGCATCATTGG<3′ (SEQ ID NO: 115), the upstream primer for gene mtn amplification, the underlined sequence was an overlapping sequence with the promoter FBA1p.
Primer 66: 5′>AAAAAAAAAAAAAAATTTTTCAGCCATCTGTTAAGAAAT TTAGCCATGTGCAAGTTTCT<3′ (SEQ ID NO: 116), the downstream primer for gene mtn amplification, the underlined sequence was an overlapping sequence with the terminator NAT5t.
Primer 67: 5′>CAAGCTATACCAAGCATACAATCAACTATCTCATATACA ATGCCGTTGTTAGATAGCTT<3′ (SEQ ID NO: 117), the upstream primer for gene luxS amplification, the underlined sequence was an overlapping sequence with the promoter ADH1p.
Primer 68: 5′>TGAAAAAAAAAAGTGGTAGATTGGGCTACGTAAATTCGA CTAGATGTGCAGTTCCTGCA<3′ (SEQ ID NO: 118), the downstream primer for gene luxS amplification, the underlined sequence was an overlapping sequence with the terminator IDP1t.
2) The amplification was conducted with the following corresponding primers using the genome of Saccharomyces cerevisiae as a template, to obtain promoters: FBA1p (nucleotide sequence shown in SEQ ID NO: 18), ADH1p (nucleotide sequence shown in SEQ ID NO: 9); terminators: NAT5t (nucleotide sequence shown in SEQ ID NO: 38), IDP1t (nucleotide sequence shown in SEQ ID NO: 39). The primers 55 and 74, PCR-amplified HIS screening marker gene, and the gene fragments amplified in the above step were assembled into gene expression cassettes according to the processes a to b in step 3) of Example 1: HIS-FBA1p-mtn-NAT5t-ADH1p-luxS-IDP1t. Using primers 73 and 64, the gene expression cassette assembled in Example 3: ENO2p-SAM2-SCW4t template was re-amplified to obtain a new gene expression cassette: ENO2p-SAM2-SCW4t. The above two recombinant gene expression cassettes were transformed into the genomic rDNA sites of Z2 and Z3 strains using the method of step 5) in Example 1, such that an SAH hydrolysis pathway derived from E. coli optimized the regeneration ability of SAM in Saccharomyces cerevisiae, and Z5 and Z8 strains were formed, respectively.

The gene sequence of strain Z5 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-FBA1p-pAMT-ATP15t
rDNA:HIS-FBA1p-mtn-NAT5t-ADH1p-luxS-IDP1t-ENO2p-SAM2-SCW4t

The gene sequence of strain Z8 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-THS1p-pAMT-MDM35t
Delta1:LEU-HXT7p-TAL-PRS28At-PFY1p-pheA-PRC1t-HXT7p-Aro9-BAN4t.
rDNA:HIS-FBA1p-mtn-NAT5t-ADH1p-luxS-IDP1t-ENO2p-SAM2-SCW4t.

The primer sequences involved were: (both promoter and terminator were based on the Saccharomyces cerevisiae genome as a template; the special parts were marked below):
Primer 69: 5′>ATACCGCACAGATGCGTAAGGAGAAAATACCGCATCAGG ATAACAATACTGACAGTACT<3′ (SEQ ID NO: 119), the upstream primer for the amplification of the promoter FBA1p, the underlined sequence was an overlapping sequence with the HIS screening marker gene.
Primer 70: 5′>AACTTCTTCTTCCATTGCACCAATGATGCCGATTTTCAT TTTGAATATGTATTACTTGG<3′ (SEQ ID NO: 120), the downstream primer for the amplification of the promoter FBA1p, the underlined sequence was an overlapping sequence with the gene mtn.
Primer 71: 5′>CAGGTGCAGGCTGACCTAATGGAATTTTTATGGTCCCGA ACATGTAGGTGGCGGAGGGG<3′ (SEQ ID NO: 121), the upstream primer for the amplification of promoter ADH1p, the underlined sequence was an overlapping sequence with the terminator NAT5t.
Primer 72: 5′>CCGGGTATGATCGACTGTGAAGCTATCTAACAACGGCAT TGTATATGAGATAGTTGATT<3′ (SEQ ID NO: 122), downstream primer for ADH1p promoter amplification, the underlined sequence was an overlapping sequence with the gene luxS.
Primer 73: 5′>CACCGTTTTCTTGAAACCAAACACCGAACTCACTACGCA ACGCGGCGTTATGTCACTAA<3′ (SEQ ID NO: 123), the upstream primer for the amplification of the promoter ENO2p-2, the underlined sequence was an overlapping sequence with the terminator IDP1t.
Primer 74: 5′>GCCAAGTAGGCAATTATTTAGTACTGTCAGTATTGTTAT CCTGATGCGGTATTTTCTCC<3′ (SEQ ID NO: 124), the downstream primer for amplifying the HIS screening marker gene, the underlined sequence was an overlapping sequence with the promoter FBA1p. (Taking the HIS screening marker plasmid purchased from BIOFENG as a template)
Primer 75: 5′>ATGGTTGAGTCACTGGTGCAGAAACTTGCACATGGCTAA ATTTCTTAACAGATGGCTGA<3′ (SEQ ID NO: 125), the upstream primer for the amplification of the terminator NAT5t, the underlined sequence was an overlapping sequence with the gene mtn.
Primer 76: 5′>TCTGTTCTATTGTATATCTCCCCTCCGCCACCTACATGT TCGGGACCATAAAAATTCCA<3′ (SEQ ID NO: 126), downstream primer for terminator NAT5t amplification, the underlined sequence was an overlapping sequence with the promoter ADH1p.
Primer 77: 5′>GCACTGCCGAAAGAGAAGTTGCAGGAACTGCACATCTAG TCGAATTTACGTAGCCCAAT<3′ (SEQ ID NO: 127), the upstream primer for amplification of the terminator IDP1t, the underlined sequence was an overlapping sequence with the gene luxS.
Primer 78: 5′>CGCAAGTTGGTGCACGTCGTTAGTGACATAACGCCGCGT TGCGTAGTGAGTTCGGTGTT<3′ (SEQ ID NO: 128), the downstream primer for amplification of the terminator IDP1t, the underlined sequence was an overlapping sequence with the promoter ENO2p.
3) The strains Z5 and Z8 were subjected to fermentation culture using the method of step 7) in Example 1, and the yields of vanillylamine were increased to 11.88 g/L and 16.48 g/L, which showed increases of 84% and 160% on the vanillylamine compared with the unoptimized strains, respectively.

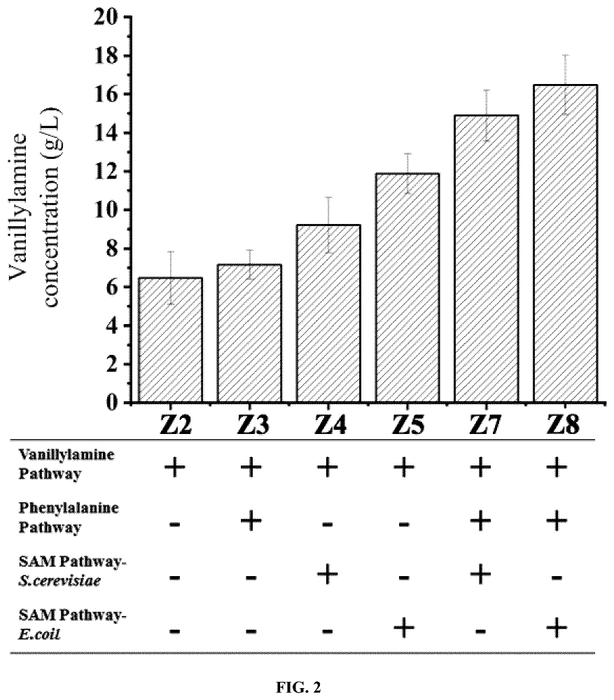

The comparative results of the vanillylamine yields of the engineered Saccharomyces cerevisiae strains obtained in Examples 1 to 4 were shown in FIG. 2.

### Example 5

Construction of engineered Saccharomyces cerevisiae strains for capsaicin synthesis
1) According to the method in step 2) of Example 1, two head-to-head promoter modules were constructed: pBM16A-PGK1p+FBA1p and pBM16A-GPM1p (nucleotide sequence shown in SEQ ID NO: 40)+ACT1p. Two back-to-back terminator modules were chemically synthesized by GENEWIZ: ASP3t (nucleotide sequence shown in SEQ ID NO: 41)-HAP4t (nucleotide sequence shown in SEQ ID NO: 49), and YCP4t (nucleoside sequence shown in SEQ ID NO: 50)--MDM35t (nucleotide sequence shown in SEQ ID NO: 42). The A22site1 plasmid (nucleotide sequence shown in SEQ ID NO: 43) of the upper sequence of YORWΔ22 site and the A22site2 plasmid (nucleotide sequence shown in SEQ ID NO: 44) of the lower sequence of YORWΔ22 site, containing G-418 resistance, were selected; the Δ22site1 and Δ22site2 plasmids were prepared using the c to g processes of step 2) in Example 1.
2) PCR amplification was conducted on the promoter module, and functional genes Kas (nucleotide sequence shown in SEQ ID NO: 45), Acl (nucleotide sequence shown in SEQ ID NO: 46), Fat (nucleotide sequence shown in SEQ ID NO: 47), and AT3 (the nucleotide sequence shown in SEQ ID NO: 48). Two double transformation units were assembled using the Golden Gate method: DH5α/PUC57T-(ASP3t-Kas-PGK1p-FBA1p-Acl-HAP4t) and DH5c/PUC57T-(YCP4t-Fat-GPM1p-ACT1p-AT3-MDM35t). Through method of step 5) in Example 1, the four genes were constructed on the genome of the Z2 strain to realize heterologous expression of the capsaicin pathway in Saccharomyces cerevisiae. The resulting strain was named C1.

The gene sequence of strain C1 was based on the genome of Saccharomyces cerevisiae:
YORWΔ15:URA3-TPIt-PAL2-TDH3p-ADH1p-C4H-PGI1t-ADH1t-4CL2-PGKp-TEF2p-HCT-CYC1t
YORWΔ17:TEF1p-hphNT1-CYC1t-HOG1t-CCoAoMT1-ENO2p-PYK1p-FerB2-LRP1t-FBA1p-pAMT-ATP15t
YORWΔ22:TEF1p-KanMX-TEF1t-ASP3t-Kas-PGK1p-FBA1p-Acl-HAP4t-YCP4t-Fat-GPM1p-ACT1p-AT3-MDM35t

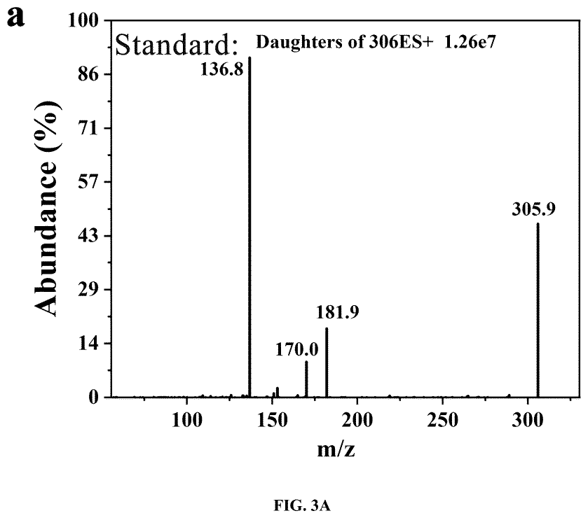

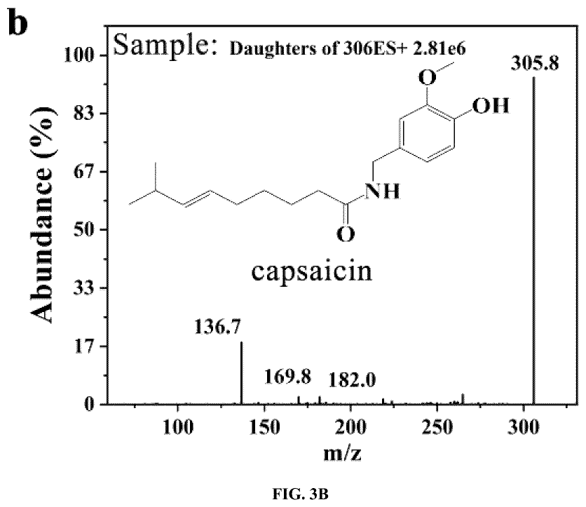

The primer sequences involved in the process were as follows: (the promoter used the Saccharomyces cerevisiae genome as a template; the gene used a synthetic plasmid as a template)
Primer 79: 5′>CCTGCTCTTCACATTGTTTTATATTTGTTGTAAAAAGTAG<3′ (SEQ ID NO: 129), the upstream primer for amplification of the promoter PGK1p, the underlined sequence was a cohesive end formed by the digestion of the promoter PGK1p by Sap1.
Primer 80: 5′>GCCAAGTAGGCAATTATTTAGTACTGTCAGTATTGTTAT ACGCACAGATATTATAACAT<3′ (SEQ ID NO: 130), the downstream primer for the amplification of the promoter PGK1p, the underlined sequence was an overlapping sequence with the promoter FBA1p.
Primer 81: 5′>GCAAATGCCTATTGTGCAGATGTTATAATATCTGTGCGT ATAACAATACTGACAGTACT<3′ (SEQ ID NO: 131), the upstream primer for the amplification of the promoter FBA1p, the underlined sequence was an overlapping sequence with the promoter PGK1p.
Primer 82: 5′>CCTGCTCTTCATGTTTTGAATATGTATTACTTGGTTATG<3′ (SEQ ID NO: 132), the downstream primer for the amplification of promoter FBA1p, the underlined sequence was a cohesive end formed after the promoter FBA1p was digested by Sap1.
Primer 83: 5′>CCTGCTCTTCACATATTGTAATATGTGTGTTTGTTTGG<3′ (SEQ ID NO: 133), the upstream primer for the amplification of promoter GPM1p, the underlined sequence was a cohesive end formed after the promoter GPM1p was digested by Sap1.
Primer 84: 5′>ATTATATTATATGGGTCTGCAAGGTAGAGGCGCGCTTGT CACATGCAGTGATGCACGCG<3′ (SEQ ID NO: 134), the downstream primer for the amplification of promoter GPM1p, the underlined sequence was an overlapping sequence with the promoter ATC1p.
Primer 85: 5′>ATGTAACTTAGCACCATCGCGCGTGCATCACTGCATGTG ACAAGCGCGCCTCTACCTTG<3′ (SEQ ID NO: 135), the upstream primer for the amplification of the promoter ATC1p, the underlined sequence was an overlapping sequence with the promoter GPM1p.
Primer 86: 5′>CCTGCTCTTCATGTTGTTAATTCAGTAAATTTTCGATC<3′ (SEQ ID NO: 136), the downstream primer for the amplification of promoter ATC1p, the underlined sequence was a cohesive end formed after the promoter ATC1p was digested by Sap1.
Primer 87: 5′>CCTGCTCTTCAGGT TTATGGCTTGTATGGGGCGAAGAC<3′ (SEQ ID NO: 137), the upstream primer for gene Kas amplification, the underlined sequence was a cohesive end of gene Kas after Sap1 digestion.
Primer 88: 5′>CCTGCTCTTCAGTA ATGTCTTCTATCACTTCTTCTTG<3′ (SEQ ID NO: 138), the downstream primer for gene Kas amplification, the underlined sequence was a cohesive end of gene Kas after Sap1 digestion.
Primer 89: 5′>CCTGCTCTTCAACA ATGGCTTCTATCACTGCTTCTTCTTTC<3′ (SEQ ID NO: 139), the upstream primer for gene Acl amplification, the underlined sequence was a cohesive end formed after gene Acl was digested by Sap1.
Primer 90: 5′>CCTGCTCTTCAGAT TTAACACTTCTTAGCAACCAAGTCTTC<3′ (SEQ ID NO: 140), downstream primer for gene Acl amplification, the underlined sequence was a cohesive end of gene Acl after Sap1 digestion.
Primer 91: 5′>CCTGCTCTTCAGGTTTAGATTCTAGATGGCTTCTTTC<3′ (SEQ ID NO: 141), the upstream primer for gene Fat amplification, the underlined sequence was a cohesive end of gene Fat after Sap1 digestion.
Primer 92: 5′>CCTGCTCTTCAGTAATGTTGTCTAGAGGTTCTTTCTTG<3′ (SEQ ID NO: 142), the downstream primer for gene Fat amplification, the underlined sequence was a cohesive end of gene Fat after Sap1 digestion.
Primer 93: 5′>CCTGCTCTTCAACAATGGCTTTCGCTTTGCCATCTTC<3′ (SEQ ID NO: 143), the upstream primer for geneAT3 amplification, the underlined sequence was a cohesive end of geneAT3 after Sap1 digestion.
Primer 94: 5′>CCTGCTCTTCAGATTTAAGCGATGAATTCAACCAATTC<3′ (SEQ ID NO: 144), the downstream primer for gene AT3 amplification, the underlined sequence was a cohesive end of gene AT3 after Sap1 digestion.
3) The strain C1 was subjected to fermentation culture using the method of step 7) in Example 1, and the culture time was extended to 120 h. After the fermentation, 100 mL of a fermentation broth was subjected to centrifugation at 5000 rpm for 5 min to collect the bacterial cells. The cells were washed twice with clean water to remove residual fermentation broth, re-centrifuged to collect the bacterial cells, 10 mL of an organic extract (ethyl acetate: n-butanol=3:1) was added to the centrifuge tube to resuspend the bacterial cells. An appropriate amount of glass beads were added, and the cells were vortexed for 30 min to ensure that the cells were fully lysed. An obtained cell lysate was re-centrifuged to obtain a supernatant, which was concentrated to 1 mL by rotary evaporation. A resulting concentrate was qualitatively and quantitatively analyzed using HPLC-MS/MS. The results were shown in FIGS. 3A-3B, and it was found that the yield of capsaicin in the strain C1 was 80.23 μg/L. In the present disclosure, the de novo biosynthesis of capsaicin in Saccharomyces cerevisiae was realized for the first time.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. An engineered Saccharomyces cerevisiae strain, comprising: a Saccharomyces cerevisiae genome comprising a first and a second gene modules inserted into the genome, wherein the first gene module (gene module 1) comprises PAL2, C4H, 4CL2, and HCT genes and the second gene module (gene module 2) comprises CCoAoMT1, FerB2, and pAMT genes, wherein said engineered strain of Saccharomyces cerevisiae produces vanillylamine.

2. The engineered Saccharomyces cerevisiae strain of claim 1, further comprising pheA, Aro9, and TAL genes inserted into the Saccharomyces cerevisiae genome.

3. The engineered Saccharomyces cerevisiae strain of claim 1, further comprising SahH and SAM2 genes inserted into the Saccharomyces cerevisiae genome.

4. The engineered Saccharomyces cerevisiae strain of claim 2, further comprising SahH and SAM2 genes inserted into the Saccharomyces cerevisiae genome.

5. The engineered Saccharomyces cerevisiae strain of claim 1, further comprising mtn, luxS, and SAM2 genes inserted into the Saccharomyces cerevisiae genome.

6. The engineered Saccharomyces cerevisiae strain of claim 2, further comprising mtn, luxS, and SAM2 genes inserted into the Saccharomyces cerevisiae genome.

7. The engineered Saccharomyces cerevisiae strain of claim 1, further comprising Kas, Acl, Fat, and AT3 genes inserted into the Saccharomyces cerevisiae genome, wherein said engineered Saccharomyces cerevisiae strain produces capsaicin.

8. The engineered Saccharomyces cerevisiae strain of claim 2, further comprising Kas, Acl, Fat, and AT3 genes inserted into the Saccharomyces cerevisiae genome.

9. The engineered Saccharomyces cerevisiae strain of claim 3, further comprising Kas, Acl, Fat, and AT3 genes inserted into the Saccharomyces cerevisiae genome.

10. The engineered Saccharomyces cerevisiae strain of claim 4, further comprising Kas, Acl, Fat, and AT3 genes inserted into the Saccharomyces cerevisiae genome.

11. The engineered Saccharomyces cerevisiae strain of claim 5, further comprising Kas, Acl, Fat, and AT3 genes inserted into the Saccharomyces cerevisiae genome.

12. The engineered Saccharomyces cerevisiae strain of claim 6, further comprising Kas, Acl, Fat, and AT3 genes inserted into the Saccharomyces cerevisiae genome.

13. A method for producing the engineered Saccharomyces cerevisiae strain of claim 1, comprising the following steps:
constructing a first double transformation unit: TPI1t-PAL2-TDH3p-ADH1p-C4H-PGI1t, and a second double transformation unit: ADH1t-4CL2-PGK1p-TEF2p-HCT-CYC1t, and inserting the first and second double transformation units into a YPRCA15 site of a genome of Saccharomyces cerevisiae to obtain a strain Z1; and
constructing a first gene expression cassette: FBA1p-pAMT-ATP15t, and a third double transformation unit: HOG1t-CCoAoMT1-ENO2p-PYK1p-FerB2-LRP1t, and inserting the gene expression cassette and the third double transformation unit jointly into a YORWΔ17 site of a genome of the strain Z1.

14. The method according to claim 13, further comprising the following steps:
inserting pheA, Aro9 and TAL genes into a genome of the engineered Saccharomyces cerevisiae strain; and
constructing a second gene expression cassette:, PFY1p-pheA-PRC1t-ACT1p-Aro9-BAN4t, and a third gene expression cassette: HXT7p-TAL-PRS28At, and inserting the second and third gene expression cassettes jointly into a delta1 site of a genome of the engineered Saccharomyces cerevisiae strain.

15. The method according to claim 13, further comprising the following steps:
inserting SahH and SAM2 genes into a genome of the engineered Saccharomyces cerevisiae strain; and
constructing a second gene expression cassette: HIS-CYC1P-SahH-ALY2t, and a third gene expression cassette: ENO2p-SAM2-SCW4t, and inserting the second and third gene expression cassettes into an rDNA site of a genome of the engineered Saccharomyces cerevisiae strain.

16. The method according to claim 13, further comprising the following steps:
inserting pheA, Aro9, TAL, SahH and SAM2 genes into a genome of the engineered Saccharomyces cerevisiae strain; and
constructing a second gene expression cassette: HIS-CYC1P-SahH-ALY2t, and a third gene expression cassette: ENO2p-SAM2-SCW4t, and inserting the second and third gene expression cassettes into an rDNA site of a genome of the engineered Saccharomyces cerevisiae strain.

17. The method according to claim 13, further comprising the following steps:
inserting mtn, luxS, and SAM2 genes into a genome of the engineered Saccharomyces cerevisiae strain; and
constructing a second gene expression cassette: HIS-FBA1p-mtn-NAT5t-ADH1p-luxS-IDP1t, and a third gene expression cassette: ENO2p-SAM2-SCW4t, and inserting the second and third gene expression cassettes into an rDNA site of a genome of the engineered Saccharomyces cerevisiae strain.

18. The method according to claim 13, further comprising the following steps:
inserting Kas, Acl, Fat, and AT3 genes into a genome of the engineered Saccharomyces cerevisiae strain; and
constructing a fourth double transformation unit: ASP3t-Kas-PGK1p-FBA1p-Acl-HAP4t, and a fifth double transformation unit: YCP4t-Fat-GPM1p-ACT1p-AT3-MDM35t, and inserting the fourth and fifth transformation units into a YORWΔ22 site of a genome of the engineered Saccharomyces cerevisiae strain.

19. A method for producing vanillylamine, comprising culturing the engineered strain of Saccharomyces cerevisae according to claim 1.

20. A method of producing capsaicin comprising culturing the engineered strain of Saccharomyces cerevisae according to claim 9.
